SEQUENCE LISTING (1 sequence)

SEQ ID NO: 1 - protein, 15 aa, Homo sapiens
MVNSSRVQPQQPGDA

(19) United States
(12) Patent Application Publication
(10) Pub. No.: US 2008/0063601 A1
(43) Pub. Date: Mar. 13, 2008

(54) Compositions and methods relating to polycystic kidney disease

(75) Inventors: Stefan Somlo, Westport, CT (US); Craig M. Crews, New Haven, CT (US); Stephanie J. Quinn, Hope Valley, RI (US); Dayne Okuhara, Lexington, MA (US)

Correspondence Address: WOLF GREENFIELD & SACKS, P.C., 600 ATLANTIC AVENUE, BOSTON, MA, 02210-2206, US

(73) Assignee: Yale University, New Haven, CT (US)

(21) Appl. No.: 11/716,980

(22) Filed: Mar. 12, 2007

Related U.S. Application Data
(63) Continuation-in-part of application No. PCT/US05/41476, filed on Nov. 15, 2005.
(63) Continuation-in-part of application No. PCT/US06/30671, filed on Aug. 9, 2006.
(60) Provisional application No. 60/627,844, filed on Nov. 15, 2004.
(60) Provisional application No. 60/707,014, filed on Aug. 9, 2005.

(51) Int. Cl. A61K 31/58; A61K 49/00; A61P 13/12; C07K 7/08; C12Q 1/02

(52) U.S. Cl. 424/9.2; 435/29; 514/171; 514/172; 530/326

(57) ABSTRACT

Described herein are therapeutic strategies (methods and compositions) useful for treating conditions in which cilia are affected and which manifest with cysts and/or fibrosis, such as conditions in which the kidney, pancreas, liver and/or spleen are affected and contain cysts. Particular embodiments described herein are therapeutic strategies in which PC-2 agonists, particularly agonists (calcium channel agonists) that target PC-2 directly and/or selectively, are administered to individuals with mutations in PKD1, in order to alter the course of polycystic kidney disease, particularly ADPKD. In specific embodiments, the invention relates to use of PC-2 agonists triptolide and triptolide derivatives to regulate calcium release. In other aspects, the invention relates to use of PC-2 agonists to treat or aid in the treatment of any condition in which a calcium channel, such as the gene product of PKD1 and/or PKD2, is mutated; calcium signaling is abnormal; or both.

### RELATED APPLICATIONS

This application claims the benefit of the filing date of U.S. Provisional Application No. 60/627,844, entitled “Triptolide treats polycystic kidney disease,” by Craig M. Crews and Stephanie J. Quinn, filed Nov. 15, 2004, and of U.S. Provisional Application No. 60/707,014, entitled “Target and Assay for Lead Compounds for Polycystic Kidney Disease,” by Stefan Somlo, filed Aug. 9, 2005. This application also claims priority to PCT application US2005/041476, entitled “Treatment of Conditions Caused by Calcium Abnormalities,” by Craig M. Crews and Stephanie J. Quinn, filed Nov. 15, 2005 and PCT application US2006/030671 entitled “Target and Assay for Lead Compounds For Polycystic Kidney Disease,” by Stefan Somlo, filed Aug. 9, 2006. The entire contents and teachings of the referenced provisional applications and the referenced PCT applications are expressly incorporated herein by reference.

### FUNDING

This invention was made with government support under Grant Number A1 055914, Grant Number DK54053, Grant Number DK57328 and Grant Number F32-DK59780, all awarded by National Institutes of Health. The government has certain rights in the invention.

### BACKGROUND OF THE INVENTION

The family of human autosomal polycystic diseases includes at least three distinct clinical entities caused by mutations in at least six different genes. These are autosomal dominant polycystic kidney disease (ADPKD; PKD1, PKD2), autosomal recessive polycystic kidney disease (ARPKD; PKHD1) and autosomal dominant polycystic liver disease without kidney cysts (ADPLD; PLD1, PLD2, PLD3). Of these cilial diseases, Autosomal Dominant Polycystic Kidney Disease (ADPKD) represents, by far, the largest public health burden. ADPKD affects between 1 in 500 and 1 in 1000 live births worldwide and is the leading genetic cause of end stage renal failure. Mutations in PKD1 (encoding polycystin-1) account for approximately 85% of all cases of ADPKD; the remaining 15% is attributed to mutations in PKD2 (encoding polycystin-2) (Igarashi and Somlo, 2002, J Am Soc Nephrol 13, 2384-2398). The mutational mechanism for cyst formation in ADPKD involves somatic acquisition of second step mutations in the normal copy of the respective gene to give rise to cysts. In addition, haploinsufficiency associated with threshold effects and trans-heterozygote second step mutations may also play a role in cyst formation. Disease progression is characterized by the consequent formation and growth of simple, fluid filled cysts derived from tubules throughout the kidney (and liver). The cellular pathogenesis of these changes is related to the inability of tubule epithelium to regulate calcium signals, which results in a loss of the fully differentiated state, increased proliferation, net fluid secretion and the formation of fluid-filled cysts in the kidney. Normal cell function and nephron structure is under the control of the mechano- and chemosensory function of primary cilia. Polycystin-1 (PC-1; also referred to as PKD1) and polycystin-2 (PC-2; also referred to as PKD2) co-localize to the primary cilia of the kidney tubule cells and bile duct cells. Polycystin-2 is a cation channel permeant to calcium (Koulen, et al., 2002, Nat Cell Biol 4, 191-197). In response to laminar flow shear stress forces, the cell's primary cilium bends and calcium enters the cell through mechanisms dependent on polycystin-2. Currently the only therapeutic intervention available to patients who develop kidney failure from polycystic kidney disease is renal replacement by either dialysis or transplantation. There are currently no therapeutic interventions available that are capable of slowing or altering the course of polycystic disease. There is great demand for therapies to improve the condition of patients with ADPKD and other cilia-based diseases.

### SUMMARY OF THE INVENTION

Described herein are therapeutic strategies (methods and compositions) useful for treating conditions which manifest with cysts, such as conditions in which the kidney, pancreas, and/or liver are affected and contain cysts. Such strategies are useful for treating, for example, ADPKD, ARPKD and other cilia-based diseases, as well as fibrocystic diseases (e.g., Bardet Biede syndrome, Meckel-Gruber, nephronophthisis and the related family of diseases in which cilia structure or function is thought to be abnormal). Particular embodiments described herein are therapeutic strategies in which PC-2 agonists, particularly agonists (calcium channel agonists) that target PC-2 directly and/or selectively, are administered to individuals with mutations in PKD1, in order to alter the course of polycystic kidney disease, such as ADPKD or ARPKD. Further embodiments are therapeutic strategies (methods and compositions) for use in other conditions which affect cilia and manifest with cysts, such as conditions which manifest with cysts and/or fibrosis of the pancreas and/or liver. These strategies can be applied, for example, to conditions in which function of cilia on bile duct cells and cilia on pancreatic duct cells are affected.

In this application, PC-1, PC-1 and PKD1 are used interchangeably to refer to polycystin-1 protein; PC-2, PC2 and PKD2 are used interchangeably to refer to polycystin-2 protein. PKD1 refers to a human gene encoding PC-1; PKD2 refers to a human gene encoding PC-2. Pkd2 and Pkd1 refer, respectively, to the mouse homolog of PKD2 and PKD1.

As described in detail below, Applicant has determined that PC-2 has endogenous cilial trafficking information within its primary (amino acid) sequence and that trafficking is independent of (does not require) PC-1. As also discussed below, Applicant observed that COOH-terminal truncated forms of PC-2 that do not interact with PC-1 still traffic to cilia and that both native and epitope tagged over-expressed full length PC-2 are expressed normally in cilia. Thus, Applicant has shown that PC-2 is available in the cilia of kidney tubule epithelial cells, even in the absence of PC-1. These findings support the conclusion that PC-2 is present in cilia of cells that line cysts in kidneys of patients who have mutations in PKD1 and, thus, have or are at risk of having ADPKD.

Applicant has shown that triptolide, a natural product from a Chinese medicinal herb, Tripterygium wilfordii hook-f, stimulates intracellular calcium release by a mechanism dependent on polycystin 2 (PC-2), a calcium channel that is mutated in polycystic kidney disease (PKD). Applicant has also demonstrated a calcium dependent effect on triptolide binding and function and that at different concentrations, triptolide either arrests cell growth or actively induces cell death via apoptosis. Further, Applicant has assessed the therapeutic efficacy of triptolide in a model for ADPKD, which is characterized by mutations in the gene product of PKD1 and/or PKD2, abnormal calcium influx and disregulated cell proliferation. Based at least in part on the results of that assessment, Applicant provides a novel method of regulating calcium influx, arresting cell growth and reducing or slowing cyst progression in conditions in which a calcium channel or channel complex, such as the product encoded by PKD1 or PKD2, is mutated and/or calcium signaling is abnormal, as well as therapeutic agents (drugs) and pharmaceutical compositions useful in the method. The methods and compositions described herein are useful for therapy (preventing, treating an existing condition) of a variety of cilia based diseases. For example, they are useful for therapy of diseases with cilia dysfunction (abnormal function), ADPKD, ARPKD and fibrocystic diseases (e.g., Bardet Biede syndrome, nephronophthisis, Meckel-Gruber) and other diseases in which cilia structure or function is thought to be abnormal.

The present invention provides a method of treating or aiding in the treatment of polycystic kidney disease (PKD) (e.g., ADPKD or ARPKD) in an individual in need thereof. Such method comprises administering to the individual a therapeutically effective amount of a (at least one; one or more) PC-2 (PKD2) agonist. As described herein, a “PC-2 (PKD2) agonist” mimics or enhances PC-2 activities, such as calcium signaling. PC-2 agonist binds to PC-2 or interacts, directly or indirectly, with PC-2 or with a molecular complex containing PC-2 to effect flux of calcium through the PC-2 channel and provide a cellular signal that is missing or reduced in individuals affected by ADPKD, ARPKD or cilia based disease conditions (diseases with cilia dysfunction). Such signals may affect, for example, cell proliferation, cell cytoskeletal properties, cell polarization and transport properties, cell apoptosis, any or all of which may be deranged in organs and tissues affected by polycystic diseases such as ADPKD or ARPKD. Optionally, the method further comprises administering to the individual a second (at least one additional) therapeutic agent for treating PKD, such as an EGF receptor kinase inhibitor, a cyclooxygenase 2 (COX2) inhibitor, a vasopressin V₂ receptor inhibitor, a ligand of a peripheral-type benzodiazepine receptor (PTBR), a somatostatin analogue (e.g., octreotide), and/or pioglitazone. A specific example of a PC-2 agonist is a triptolide-related compound. The term “triptolide-related compound,” as used herein, includes triptolide, triptolide prodrugs, and triptolide derivatives or analogs. Triptolide-related compounds include, but are not limited to, triptolide, a triptolide prodrug, and a triptolide derivative such as triol-triptolide, triptonide, 14-methyl-triptolide, 14-deoxy-14α-fluoro-triptolide, 5α-hydroxy triptolide, 19-methyl triptolide, and 18-deoxo-19-dehydro-18-benzoyloxy-19-benzoyl triptolide, and 14-acetyl-5,6-didehydro triptolide.

PC-2 agonists identified by the method described herein or by any other method are useful in methods of therapy (methods of treatment, prevention). The method comprises administering a PC-2 agonist to an individual in need thereof in sufficient quantity and by an appropriate route to cause the desired effect, such as treatment or prevention of ADPKD or ARPKD. For example, a therapeutically effective amount of one or more PC-2 agonists can be administered to an individual who has or is at risk of having ADPKD to have the desired effect of preventing ADPKD, reversing ADPKD, preventing progression of ADPKD or reducing the extent to which ADPKD occurs in the individual. This method can also be carried out to treat or prevent ARPKD and other cilia based disease conditions, such as those referred to herein. For example, a therapeutically effective amount of one or more PC2 agonists can be administered to an individual who has or is at risk of having ARKPD or other cilia based disease condition, in order to prevent, reverse or slow the progression of ARKPD or other cilia based disease condition or reduce the extent to which ARKPD or other cilia based disease condition occurs. Preferably, PC-2 agonists administered according to the present method are selective (act only or substantially only on PC-2 in kidney tubule, bile duct and pancreatic duct epithelial cells). A PC-2 agonist can be administered alone or in combination with one or more additional (different) PC-2 agonists and/or with drugs useful in treating ARPKD. The PC-2 agonist can be administered by a variety of routes, such as a route selected from oral administration, topical administration, parenteral administration, intravaginal administration, rectal administration, systemic administration, intramuscular administration and intravenous administration. PC-2 agonists used in methods of therapy for an individual who has or is at risk of having ADPKD include, but are not limited to triptolide-related compounds.

In one embodiment, the PC-2 agonist, such as a triptolide-related compound, is administered prior to the development of symptomatic renal disease in the individual such that ADPKD or ARPKD is prevented. For example, the individual has been determined to be at risk of PKD as determined by family history, renal imaging study and/or genetic screening. In another embodiment, the PC-2 agonist (e.g., a triptolide-related compound) is administered when the individual exhibits symptomatic renal disease and, as a result, the disease progression is slowed or inhibited. Preferably, the individual is a mammal such as a human. In certain cases, the PC-2 agonist is administered to an individual in combination with a surgical therapy, such as partial removal of a kidney or kidney transplant. Although not wishing to be bound by any particular mechanism or theory, it is believed that the triptolide-related compound regulates calcium signaling in kidney cyst tissues.

In certain embodiments, the present invention provides a method of treating a cystic disease in an individual in need thereof. Such method comprises administering to the individual a therapeutically effective amount of a PC-2 agonist in an amount sufficient to slow or inhibit growth of cyst cells. A PC-2 agonist can be used therapeutically for individuals with or at risk of having fibrocystic disease, such as Bardet Biede Syndrome, nephronophthisis, Meckel-Gruber and the related family of disease in which cilia structure or function is thought to be abnormal. The cystic disease includes, but is not limited to, breast cysts, bronchogenic cysts, choledochal cysts, colloidal cysts, congenital cysts, dental cysts, epidermoid inclusions, hepatic cysts, hydatid cysts, lung cysts, mediastinal cysts, ovarian cysts, periapical cysts, pericardial cysts, and polycystic kidney disease (PKD). A specific example of the PC-2 agonist is a triptolide-related compound. Preferably, the individual is a mammal such as a human.

In certain embodiments, the present invention provides a method of slowing or inhibiting cyst formation. Such method comprises contacting cyst cells with a PC-2 agonist in an amount sufficient to slow or inhibit cyst formation. The cyst cells can be, for example, kidney cyst cells present in an individual having or at risk of developing PKD (e.g., ADPKD). Preferably, the cyst cells are mammalian cells (e.g., human cells). A specific example of the PC-2 agonist is a triptolide-related compound. Other conditions for which methods of this invention are useful includes fibrocystic diseases as described herein and conditions in which cilia function and/or structure are abnormal.

In certain embodiments, the present invention provides a method of regulating calcium influx in a cell expressing polycystin-1 (PC-1) or polycystin-2 (PC-2). Such method comprises contacting the cell with an effective amount of a PC-2 agonist. The cell can be, for example, a kidney cell, such as a kidney cell present in or isolated from an individual having or at risk of developing PKD (e.g., ADPKD, ARPKD). A specific example of the PC-2 agonist is a triptolide-related compound, which includes, but is not limited to, triptolide, a triptolide prodrug, and a triptolide derivative such as triol-triptolide, triptonide, 14-methyl-triptolide, 14-deoxy-14α-fluoro-triptolide, 5α-hydroxy triptolide, 19-methyl triptolide, and 18-deoxo-19-dehydro-18-benzoyloxy-19-benzoyl triptolide, and 14-acetyl-5,6-didehydro triptolide.

In certain specific embodiments, the present invention relates to the use of a PC-2 agonist for treating or aiding in the treatment of any condition in which a calcium channel, such as the gene product of PKD1 and/or PKD2, is mutated and/or calcium signaling is abnormal. Also described herein is the use of PC-2 agonists (e.g., triptolide-related compound) to arrest (decrease, partially or completely) cellular proliferation and/or attenuate (slow, prevent or reverse) cyst formation by restoring calcium signaling in cystic cells such as those in PKD. Specifically described herein is the ability of a PC-2 agonist to arrest cellular proliferation and attenuate overall cyst formation by restoring calcium signaling in these cells.

In certain embodiments, this invention provides a method of treating or aiding in the treatment of a condition (referred to as a condition caused by calcium abnormality) in which PC-1 is mutated and/or calcium signaling is abnormal. Such method comprises administering a PC-2 agonist (e.g., a triptolide-related compound) to an individual in need of such treatment. A PC-2 agonist is administered in sufficient quantity to correct (partially or completely) the calcium abnormality and restore (partially or completely) calcium signaling, thereby treating or aiding in the treatment of the condition caused by calcium abnormality. As described herein, the phrase “restoring calcium signaling” refers to bringing calcium signaling to a level which results in reduction (slowing the rate, and reducing the extent) in or arrest of cell proliferation and attenuation (slowing, reducing extent) of cyst formation. It is not necessary that cyst formation or cyst growth be completely prevented or arrested. In fact, thinking in terms of therapeutic index, an agent given in sufficient doses to slow growth, but not arrest it, may be able to circumvent and potential side effects of larger doses. While proliferation is a good target to slow cyst growth, there are other potential targets (e.g., the ability of the cyst cells to remodel the surrounding tissues). Thus, slowing cyst growth is important, but is not limited to being done only by blocking proliferation, since other effects may also result in slowed cyst growth. In specific embodiments, such condition is PKD (e.g., ADPKD or ARPKD). In one embodiment, a PC-2 agonist is administered to an individual in sufficient quantity to regulate intracellular calcium release, particularly to restore (partially or completely) intracellular calcium release. In a further specific embodiment, a PC-2 agonist is administered to an individual in whom mutation is present in the PKD1 gene, but not in the PKD2 gene to regulate activity/function of the PKD2 gene and prevent the individual from developing PKD (e.g., ADPKD, ARPKD) or limit the extent to which PKD (e.g., ADPKD, ARPKD) occurs. In each embodiment, calcium signaling is restored to such an extent to result in arrest of cellular proliferation and/or attenuation of cyst formation Additional examples of conditions of cilia based diseases and fibrocystic diseases in which therapy as described herein can be useful include, but are not limited to, MCKD (medullary cystic kidney disease), TSC (Tuberous sclerosis), nephronophthisis, and Bardet-Biede syndrome.

In certain embodiments, one or more PC-2 agonists (e.g., triptolide-related compounds) may be administered to the individual by a variety of routes, for example, orally, topically, parenterally, intravaginally, systemically, intramuscularly, rectally or intravenously. In certain embodiments, a PC-2 agonist is formulated with a pharmaceutical carrier.

In certain embodiments, a PC-2 agonist (e.g., a triptolide-related compound) can be administered alone or in combination with each other and/or with a second agent or drug, such as an EGF receptor kinase inhibitor, a COX2 inhibitor, a vasopressin V₂ receptor inhibitor, a ligand of PTBR, a somatostatin analogue (e.g., octreotide), cell cycle dependent kinase inhibitors (CDK e.g., roscovitine), mitogen activate protein kinase (MAPK) pathway inhibitors, rapamycin and pioglitazone for treating ADPKD. Alternatively, this approach can be used to treat ARPKD or other cilia-based conditions. For example, triptolide, a precursor thereof (e.g., a prodrug) or a triptolide derivative can be administered to an individual in need of treatment, alone or in combination with each other (e.g., triptolide and/or a triptolide analogue) or with a second agent or drug (e.g., triptolide and an EGF receptor kinase inhibitor, V2 receptor antagonist, rapamycin (or other drug, such as those listed above). The second agent can be administered with a PC-2 agonist in the same formulation or in separate formulations, to enhance treatment. In these embodiments, the PC-2 agonist and the second agent can be administered at the same time (simultaneously) or at separate times (sequentially), provided that they are administered in such a manner and sufficiently close in time to have the desired effect.

Also the subject of this invention are pharmaceutical compositions useful for treating or aiding in the treatment of an individual for a condition in which there is disruption of a calcium channel function, such as the gene product of PKD1 and/or the gene product of PKD2, is mutated and/or calcium signaling is abnormal. Such compositions comprise one or more PC-2 agonists. For example, the compositions of the present inventions are useful for treatment or aiding in the treatment of PKD (e.g., ADPKD or ARPKD) in an individual in need thereof.

In further embodiments, the present invention relates to use of a PC-2 agonist in the manufacture of medicament for the treatment of a cystic disease and use of a PC-2 agonist in the manufacture of medicament for the treatment of a condition caused by abnormal calcium signaling.

Also described herein are methods and compositions relating to polycystic kidney disease (PKD), particularly autosomal dominant polycystic kidney disease (ADPKD) and autosomal recessive polycystic kidney disease (ARPKD), which are useful for identification of agonists and antagonists of PKD2 product, PC-2, and therapy. For example, the assay methods described herein can be used to identify agonists and antagonists, such as small molecules, that act on PC-2 in cells, such as PC-2 in epithelial cells, including PC-2 in kidney tubule epithelial cells, such as PC-2 in cilia present on kidney tubule epithelial cells and PC-2 in the endoplasmic reticulum of cells, such as endoplasmic reticulum of kidney tubule epithelial cells. For example, the methods described can be used to identify agonists and antagonists, such as small molecules, that act on PC-2 in the absence of PC-1 or in the presence of less than normal levels of PC-1 (e.g., in individuals in whom PC-1 levels are below a physiologically relevant threshold value). They can also be used to identify agents that enhance PC-2 activity (such as agents useful to treat individuals in whom PC-2 expression (PC-2 produced) and/or function are less than normal/compromised. In specific embodiments, the methods and compositions of the present invention are useful to identify agents which are agonists of PC-2, such as small molecules that, in effect, replace or make it possible to avoid the need for PC-1, thus making it possible for PC-2 to function, even in individuals in whom PC-1 (a receptor necessary for PC-2 activation) is deficient or absent. Such methods can be used to identify small molecules that are calcium channel agonists; are of appropriate size to be filtered at the glomerulus in the kidney and reach the tubule lumens; and act on PC-2, such as in kidney tubule lumen epithelial cells, even in the absence of PC-1 or in the presence of limited quantities of PC-1. They are particularly useful to identify small molecules that activate PC-2, which Applicant has shown is expressed in cilia, even in the absence of PC-1. Such methods make it possible to identify agents, such as small molecules, that bypass the PKD1 genetic lesion that underlies ADPKD and can be administered to an individual who has or is at risk of developing ADPKD, in order to prevent, slow the progression of and/or reverse cyst formation and, thus, prevent, slow the progression of and/or reverse ADPKD in the individual. The methods are also useful to identify agents, such as small molecules, for administration to an individual who has or is at risk of developing ARPKD, in order to prevent or slow the progression of and/or reverse the cyst formation and, thus, prevent or slow the progression of and/or reverse ARPKD in the individual.

Alternatively, methods described herein can be used to identify agents (compounds, small molecules) that enter cells and act on the PC2 channel in the endoplasmic reticulum (ER). These agents can enter from the blood/plasma and do not need to be filtered at the glomerulus. Agents identified by or useful in methods described herein are not limited to those that act on the ciliary fraction of PC-2 only. Agents that act directly on PC-2 present in the ER may be used in therapy of conditions described herein.

In specific embodiments, the invention is a method of identifying agonists that act on PC-2 in kidney tubule epithelial cells (e.g., in endoplasmic reticulum, cilia of such cells). In a representative embodiment, the assay method assesses calcium increase in cells in which PC-2 is expressed and PC-1 is deficient (cells which lack adequate levels of functional PC-1, including cells in which PC-1 is not expressed). Calcium (e.g., level or concentration) can be monitored, for example, by assessment of calcium sensitive fluorescent dye loaded into the cells. In a specific embodiment, the invention is a method of identifying a PC-2 agonist, the method comprising (a) contacting cells that express PC-2 and lack adequate levels of (are deficient in) functional PC-1 with a candidate PC-2 agonist, under conditions appropriate for PC-2 mediated increase in calcium inside the cells to occur; (b) assessing calcium (e.g., by determining level, concentration) inside the cells; (c) comparing calcium in the cells as assessed in (b) with calcium in corresponding cells, under the same conditions as in (a) but in the absence of the candidate PC-2 agonist, wherein if there is a greater increase in calcium inside the cells in (a) than in the corresponding cells in the absence of the candidate PC-2 agonist (e.g., if the level or concentration of calcium in cells is greater in cells in the presence of the candidate PC-2 agonist than in its absence), the candidate PC-2 agonist is a PC-2 agonist.

In a second embodiment of the method of identifying a PC-2 agonist, particularly a selective PC-2 agonist, the same procedure as described above is carried out except that two types of cells are cultured in the presence of the candidate agonist—one that expresses PC-2 and lacks adequate levels of functional PC-1 and one that does not express PC-2 and lacks adequate levels of functional PC-1. These are also referred to, respectively, as cells that express PC-2 and cells that do not express PC-2. If cells that express PC-2 show an increase in cellular calcium and cells that do not express PC-2 fail to respond (do not show an increase or show an insubstantial increase in cellular calcium), the candidate PC-2 agonist is a PC-2 agonist, particularly a selective PC-2 agonist. In this embodiment, the method of identifying a PC-2 agonist, such as a selective PC-2 agonist, comprises: (a) contacting cells that express PC-2 and lack adequate levels of (are deficient in) functional PC-1 with a candidate PC-2 agonist, under conditions appropriate for PC-2 mediated increase in calcium inside the cells to occur; (b) assessing calcium (e.g., by determining level, concentration) inside cells contacted in (a); (c) contacting cells that do not express PC-2 and lack adequate levels of (are deficient in) functional PC-1 with a candidate PC-2 agonist, under conditions appropriate for PC-2 mediated increase in calcium inside the cells to occur (under the same conditions as in (a)); (d) assessing calcium (e.g., by determining level, concentration) inside cells contacted in (c) and (e) comparing calcium in cells assessed in (b) with calcium in cells assessed in (d), wherein if there is a greater increase in calcium inside the cells contacted in (a) (in cells that express PC-2) than in the cells contacted in (c) (in cells that do not express PC-2; e.g., if the level or concentration of calcium in cells is greater in cells that express PC-2 than in cells that do not express PC-2), the candidate PC-2 agonist is a selective PC-2 agonist.

Cells that express PC-2 and lack adequate levels of functional PC-1 and cells that do not express PC-2 and lack adequate levels of functional PC-1 used in the methods of the invention are of the same type and are contacted with the candidate agonist under the same conditions. It is not necessary that the method be carried out at the same time with both types of cells. Rather, results can be compared with a preexisting standard or reference, such as the results of an earlier assay in which the conditions were the same. Cells that do not express endogenous PC-2 and lack adequate levels of functional PC-1 but are altered or modified in such a manner that they express heterologous PC-2 or PC-2 encoded by an activated, normally silent gene, should respond to a candidate agonist in a manner similar to that in which cells expressing endogenous PC-2 respond. The method of identifying a selective agonist described above can further comprise altering or modifying cells that do not express endogenous PC-2 (e.g., cells that are the same type of cells as those that express endogenous PC-2) such that they express PC-2 (e.g., heterologous PC-2 or PC-2 encoded by a normally silent gene that has been activated) and assessing the effect of the candidate agonist on calcium in the cells. Such cells should respond to the candidate agonist in substantially the same manner as corresponding cells that express endogenous PC-2. Cells that express PC-1 but do not express adequate levels of PC-2 or lack PC-2 expression can also be used in the method. Cells which express PC-1 but lack PC-2 or express inadequate levels of PC-2 will not respond to a candidate PC-2 agonist.

The method can be carried out in a variety of formats and, in one embodiment, is a high throughput assay in which cells (e.g., cells that express PC-2, but lack adequate levels of functional PC-1) are grown to confluence and allowed to form cilia, such as in 96-well and higher density formats. Since the cells may be conditionally immortalized using a temperature sensitive SV40 large T antigen transgene, the cells will be plated in a high density format and grown under non-permissive conditions (that do not allow expression of the immortalizing transgene) until they are confluent, polarized and have formed cilia. These conditions are empirically determined and used consistently for the high throughput assay. The conditions for high throughput loading of the cells with a calcium sensitive fluorescent dye will also be empirically determined to ensure uniform loading and then applied consistently in the assay procedure. Detection methods and systems that can detect calcium entry into cells in the format used, such as an imaging system that can screen for fluorescence changes in a high throughput format, can be used. For example, the detection method can be used to screen for calcium sensitive fluorescent dye that has been introduced into cells used in the assay, such as cells (e.g., kidney tubule epithelial cells) that express PC-2, but lack adequate levels of functional PC-1. In one embodiment, an increase in fluorescence (which indicates increase of calcium inside cells) indicates that a candidate being tested is a PC-2 agonist and can be further validated by methods described in the previous sections. A wide variety of dyes, such as Fluo-4-AM, that increase in fluorescence in response to calcium, can be used. Alternatively, radiometric dyes, such as Fura-2, Fura Red™ and Indo-1, which change their excitation wavelength upon binding calcium, can be used, particularly for measuring concentration or level of calcium (quantitative measurements).

In further embodiments, the methods are useful to identify agents, such as small molecules, useful in therapy of other conditions in which an individual has a cilia based disease or a fibrocystic disease (e.g., cysts in another organ(s), such as the liver, and/or pancreas) that arise from loss or inadequate expression of PKD1. In addition, such compounds may be useful in treating other diseases of cilia structure and function, such as the manifestations of these diseases in the kidney or liver. Examples may include ARPKD and other conditions described herein.

Candidate PC-2 agonists can be obtained from a variety of sources, such as libraries of compounds that are available commercially or can be made using known methods and collections of naturally-occurring compounds. For example, candidate agonists can be any chemical and can be made synthetically or by recombinant techniques or isolated from a natural source. They can be peptides, polypeptides, proteins, sugars, hormones or nucleic acids. The candidates can be small molecules or more complex molecules, such as those made using combinatorial methods and combined into libraries. The candidates can also be natural or genetically engineered products isolated from lysates or growth media of cells, such as animal, plant or bacterial cells. Individual candidates can be tested as well.

Any cell type in which PC-2 is expressed and that lacks adequate levels of functional PC-1 or that can be altered in such a manner that PC-2 is expressed and adequate levels of functional PC-1 are lacking can be used in the method. Alternatively, any cell type in which PC-2 is not expressed and that lacks adequate levels of functional PC-1 can be used. Typically, epithelial cells and particularly kidney tubule epithelial cells, such as kidney tubule epithelial cells that have formed cilia, are used in the method. In particular embodiments, kidney epithelial cell lines that lack adequate levels of functional PC-1 (are deficient in PC-1) and express PKD2 (express PC-2); kidney epithelial cell lines that lack adequate levels of PC-2 and express PKD1 (express PC-1) or kidney epithelial cell lines that lack adequate levels of both PC-1 and PC-2 are used. Such cell lines can be made from Pkd1 and Pkd2 conditional mutant mouse lines, such as those described by Wu and co-workers (see, reference 61) and mouse cell lines carrying floxed alleles of Pkd1 and Pkd2, the mouse homologs of PKD1 and PKD2. See, for example, Piontek et al., J. Amer. Soc. Nephrol; Grimm, D H et al. JBC, 2003. Cell lines can be further altered, for example, by re-expressing wild type or mutant forms of PC-2 in cell lines lacking sufficient expression of endogenous PC-2 but expressing PC-1 or cell lines lacking sufficient expression of PC-2 and sufficient expression of endogenous PC-1. Mutant forms of PC-2 re-expression include the naturally occurring patient variant with a single amino acid substitution that lacks channel activity (D511V; Koulen et. al. Nature Cell Biol, 2002) or an engineered mutant that has channel activity but cannot traffic to cilia due to substitution at the R₆VXP motif required for cilia location (Geng et al. J. Cell Sci, 2006).

Agonists identified through the use of the methods described herein can be further assessed for their ability to activate PC-2 (for their activity as PC-2 agonists) in vivo. For example, they can be tested in a suitable animal model, such as a mouse model, in which the potential clinical effect can be evaluated. Such an animal model (e.g., a mouse model) can be, for example, models, such as conditional models, of PKD, particularly models of ADPKD or ARPKD. In these models, the homologs of the human PKD-1 and PKD-2 genes are altered, such as to produce animals in which the PKD-1 homolog is not expressed or is expressed at low levels, animals in which the PKD-2 homolog is not expressed or is expressed at low levels or animals in which neither homolog is expressed or is expressed at low levels. In these animal models, conditional inactivation of Pkd1 or Pkd2 or both genes together can be achieved in a tissue restricted manner (e.g. selected tubules of the kidney) and/or in a temporally controlled manner (e.g., in embryonic, early postnatal or adult mice). Treatment can begin before, during or after cysts have begun to form in the kidney or liver. Pre-clinical efficacy of the treatment can be assessed by a range of readouts including serum markers of kidney function (e.g. creatinine, blood urea nitrogen), by kidney volume, by microscopically assessed cystic index determined by the area of kidney sections occupied by cystic compared to non-cystic parenchyma, by kidney weight and kidney weight normalized to total body weight, or by non-invasive imaging techniques that can assess cystic index and kidney volume (e.g. small animal ultrasonography, small animal magnetic resonance imaging).

In addition, agonists identified through methods described herein can be assessed for their effectiveness as agonists in other contexts, such as in cells in which a calcium channel protein other than PC-2 is central to a condition which manifests itself by cyst formation and/or fibrosis. For example, they can be assessed using cells that serve as models or appropriate test systems for conditions of the liver or pancreas in which cysts and/or fibrosis are characteristic.

Also the subject of the present invention are pharmaceutical compositions comprising one or more PC-2 agonists. Such pharmaceutical compositions can additionally comprise drugs that are not PC-2 agonists (such as those described above) and/or inactive components, such as buffering agents, flavorants and excipients.

Also described herein is a motif R₆VXP, which is at the NH₂-terminus of PC-2, that is necessary for cilial localization of PC-2. Further the first 15 amino acids of PC-2 have been shown to be sufficient to localize to cilia heterologous proteins that do not normally traffic to cilia. One embodiment of this invention is a peptide that comprises the first 15 amino acids of PC-2 or an equivalent peptide. An equivalent peptide is a peptide in which at least one component amino acid is different from the amino acid in the corresponding position in the first 15 amino acids of PC-2, but has substantially the same ability to traffic heterologous proteins to cilia as the peptide comprising the first 15 amino acids of PC-2. Such a peptide can be used, for example, to deliver or target a heterologous protein or other moiety (e.g., a diagnostic agent, such as an imaging agent; a therapeutic agent) to cilia, such as cilia on kidney tubule epithelial cells. In one embodiment, this invention relates to peptides, referred to as trafficking peptides, that comprise the first 15 amino acid residues of PC-2. The first 31 amino acid residues of PC-2 from several species are shown in FIG. 1. In one embodiment, the first 15 amino acid residues that comprise a trafficking peptide are those present in human PC-2: MVNSSRVQPQQPGDA (SEQ ID NO. 1). Equivalent peptides of this trafficking peptide comprise at least one alteration in the 15 amino acid residues represented and have substantially the same ability to traffic heterologous proteins to cilia as the peptide comprising the first 15 amino acids of human PC-2. Such peptides are useful to traffic or deliver proteins or other moieties to cilia on kidney tubule epithelial cells. For example, they can be used to deliver or target therapeutic agents, such as calcium channel agonists (in specific embodiments, PC-2 agonists) to cilia in individuals who have ADPKD, in order to treat the condition and, as a result, prevent the progression of the condition or reverse the extent to which it exists. Alternatively, trafficking peptides of the invention can be used to deliver or target therapeutic agents to cilia in individuals at risk of developing ADPKD. As a result of this prophylactic use of trafficking peptides, ADPKD does not develop in individuals or develops to a lesser extent than would be the case if the trafficking peptides were not administered. The motif R₆VXP is necessary for cilial localization of PC-2. In specific embodiments, trafficking peptides comprise the motif R₆VXP, shown to be necessary for cilial localization of PC-2. In these embodiments, a peptide comprising the first 15 amino acid residues of human PC-2 or an equivalent peptide can be represented as: JJJJJRVXPJJJJJJ (SEQ ID NO. 2), in which each J represents an amino acid residue whose presence in the peptide results in or does not interfere with the peptide's cilial localization activity. Some or all of the constituent amino acid residues can be independently changed. For example, each amino acid residue represented by J can be that which occurs in the corresponding position in a PC-2 peptide represented in FIG. 1A or can be replaced by an amino acid other than that represented in FIG. 1A. There can be, for example, conserved amino acid substitutions.

### BRIEF DESCRIPTION OF THE DRAWINGS

The file of the patent contains at least one drawing executed in color. Copies of this patent with color drawing(s) will be provided by the Patent and Trademark Office upon request and payment of the necessary fee.

FIG. 1A-1E: RVXP is necessary for cilial targeting of PC-2. FIG. 1A: Alignment of the first 31 amino acids of PC-2 from several species. *, location of mutations in the most conserved residues whose cilial trafficking properties are shown in FIG. 1B-1E. +, other mutation sites tested that had no effect on cilial location. Identical residues are highlighted and boxed; residues in red abrogated cilial location of PC-2-L703X. Amino acid substitution S4A (FIG. 1B) does not affect trafficking of PC-2-L703X to cilia. Substitutions R6G (FIG. 1C) V7A (FIG. 1D) and P9A (FIG. 1E) result in loss of cilial localization of PC-2-L703X. Green, anti-HA; red, anti-acetylated α-tubulin. Scale bar, 10 μm.

FIG. 2: Schematic representation of the expression constructs used in this study. PC-2 sequences are shown in blue; PKD2L1 in red; hTFR in gray; transmembrane domains in black.

FIG. 3A-3B shows triptolide analogs and their structural dependence to compete for binding. FIG. 3A: Structures of triptolide and the analogs used in this study. FIG. 3B: HeLa cells were treated with [³H]-triptolide for one hour in all samples after the addition of 10 μM or 1 μM of triptolide, one of its analogs, or no competition also for one hour. Cells were washed, total cell lysates prepared, and samples were counted for bound [³H]-triptolide activity, n=3. CPM=Counts per minute by scintillation counting.

FIG. 4A-4E shows that triptolide binding is specific, membrane localized, and saturable. FIG. 4A: HeLa cells were treated with [³H]-triptolide for one hour in all samples. Competition of the radioligand was assessed by the addition of 1 μM unlabeled triptolide (cold) for one hour either before or after [³H]-triptolide addition, n=3. CPM=Counts per minute by scintillation counting. FIG. 4B: HeLa cells were labeled with [³H]-triptolide and cellular fractions were prepared. Binding was assessed as total CPM in the cytosolic (S-100), membrane (P-100), or insoluble cellular fractions. FIG. 4C: HeLa cells were labeled with [³H]-triptolide for one hour followed by preparation of total cellular lysates and addition to DEAE anion exchange resin. The resin was washed in batch elutions of increasing salt concentration following removal of the flow-through (FT). Each eluant was subsequently counted for [³H]-triptolide activity, n=3. FIG. 4D: [³H]-triptolide labeled HeLa cell P-100 fractions were run out on 8% reducing or native PAGE. Gel slices were removed utilizing molecular weight marker designations, crushed and eluted in water, and then counted for [³H]-triptolide activity by scintillation counting, n=2. FIG. 4E: HeLa cells were pre-incubated with 2 μM unlabeled triptolide or DMSO followed by increasing nanomolar concentrations of [³H]-triptolide for 1 hour. Specific binding was measured from scintillation counting of cellular lysates and receptor saturation was achieved. Non-specific binding (NSB) is shown in inset and does not reach saturation.

FIG. 5A-5C shows that extracellular calcium regulates triptolide mediated binding and cell death induction. FIG. 5A: HeLa cells were cultured in the presence or absence of calcium containing media for 16 hours before the addition of 30 nM [³H]-triptolide to assess binding affinity, n=3. CPM=Counts per minute. FIG. 5B: HeLa cells were cultured in the presence (+Ca²⁺) or absence (−Ca²⁺) of calcium containing media over a time course of 72 hours to determine the rate of growth in each condition, n=3.

FIG. 5C: HeLa cells were cultured in the presence (+Ca²⁺) or absence (−Ca²⁺) of calcium containing media plus 100 nM triptolide over a time course of 72 hours. Cells were washed with PBS, photographed, and counted using trypan blue at 0, 24, 48, and 72 hours to assess viability. Results are representative of three separate experiments.

FIG. 6A-6C shows that buffering cytosolic calcium can temporarily rescue triptolide induced cell death. FIG. 6A: HeLa cells were cultured in the presence of calcium containing media and transfected with one of the following constructs for 24 hours: GFP vector, NLS-parvalbumin (PV)-GFP, or NES-PV-GFP. Images were acquired by confocal microscopy (40×). FIG. 6B: Normal cell growth was assessed with each transient transfection construct, n=3. FIG. 6C: 100 nM triptolide was added to all transfected cells and viability was assessed after 24 hours, n=3.

FIG. 7A-7B shows that inhibition of NFκB transactivation is independent of the presence of calcium. FIG. 7A: HeLa cells were transfected with a κB-luciferase construct for all experimental conditions for 24 hours before the addition of 15 ng/ml TNF-α±100 nM triptolide. Cells were grown in the presence or absence of calcium containing media for 16 hours before treatment and then harvested for the assay after 6 hours, n=4. FIG. 7B: Cells were transfected with one of the following: GFP vector, NES-PV-GFP, or NLS-PV-GFP at the same time as the κB-luciferase construct and treated as described in (A), n=4.

FIG. 8A-8B shows that triptolide concentration differentially effects viability/growth or NFκB Inhibition. FIG. 8A: HeLa were plated at an initial concentration of 5×10⁵ and allowed to grow ±triptolide (10-100 in M) for 48 hours. Viable (adherent) cells were photographed under 25× brightfield microscopy and cell death was assessed by trypan dye exclusion. Results are representative of 3 separate experiments. FIG. 8B: Following a transient transfection with the κB-luciferase reporter construct, HeLa cells were incubated with 15 ng/ml TNF-α±triptolide (10-100 nM) for a total of 6 hours before assessing reporter activity, n=4.

FIG. 9A-9C shows structural divergence of biological functions of triptolide analogs. All experiments were done with HeLa cells, where cell viability was measured 24 hours after the addition of triptolide or one of its analogs at concentration ranges of 0.1-10 μM. Cell viability was assessed by trypan dye exclusion and recorded as the % change in cell number from the time of triptolide or analog addition. Control cells were allowed to grow in media alone and represent a normal population doubling, n=3. NFκB Inhibition was assessed following transfection with the κB-luciferase reporter construct and treatment with triptolide or one of its analogs (0.1-10 μM) and TNF-α for 5 hours. Control represents transfected cells without TNF-α addition, n=4. FIG. 9A: Triptolide (1). FIG. 9B: Triol-Triptolide (2). FIG. 9C: Triptonide (3).

FIG. 10A-10E shows that triptolide induces a polycystin-2 dependent calcium release in murine kidney epithelial cells. FIG. 10A-C: Cells were loaded with Fluo-4 and assessed for calcium release by fluorescence intensity before and after 100 nM triptolide addition. Cell lines tested included (A) Pkd1^{-/-} (B) Pkd2^{-/-} and (C) Re-expression (Rex) of Pkd2 in the Pkd2^{-/-} background. FIG. 10D: The average change in fluorescence amplitude was calculated from baseline levels (n=44 or 66 for Pkd2^{-/-} or Pkd2 Rex). FIG. 10E: Western blot analysis of polycystin-2 expression in each of the cell lines tested.

FIG. 11A-11J shows that Pkd1^{-/-} murine kidney epithelial cells undergo growth arrest and p21 upregulation upon triptolide treatment. FIG. 11A-E: Pkd1^{-/-} cells were treated with 100 nM triptolide over a time course of 96 hours. Representative fields were photographed under brightfield microscopy (10×). FIG. 11F: Pkd1^{-/-} triptolide treated cells after 96 hours showing a flattened morphology (40×). FIG. 11G: Confocal microscopy of Pkd1^{-/-} cells for polycystin-2 immunofluorescent expression (FITC, 40×). FIG. 11H: Western blot analysis of p21 and FIG. 1: active caspase-3 expression in Pkd1^{-/-} cells during a time course of 100 nM triptolide treatment. FIG. 11J: Viable cells were counted by the method of trypan blue dye exclusion in Pkd1^{-/-} (Mean ±SE, n=5) and Pkd2^{+/-} (n=8) cells over a time course with 100 nM triptolide addition.

FIGS. 12A-12N shows that triptolide reduces cystic burden in a Pkd1^{-/-} murine model of polycystic kidney disease. (A-C) Representative kidneys from Pkd1^{-/-} pups treated with DMSO during gestation (E10.5-birth). Large cysts are present throughout the medulla and cortex (10× magnification). (D-F) Representative kidneys from Pkd1^{-/-} pups treated with triptolide during gestation. (G) Pkd1^{+/+} kidney from a pup treated with DMSO or (H) triptolide. (I) Pkd1^{+/-} kidney treated with DMSO or (J) triptolide. (K-M) IHC staining of Pkd1^{-/-} kidneys for active caspase-3 expression: (K) secondary antibody negative control, (L) DMSO treated, (M) triptolide treated. (N) The percent of cyst burden in the kidney as determined by area in each of the Pkd1 genotypes (Mean ±SE, Pkd1^{-/-}, n=19; Pkd1^{+/-} and Pkd1^{+/+}, n=10).

### DETAILED DESCRIPTION OF THE INVENTION

As described herein, the motif, R₆VXP, at the NH2-terminus of PC-2 is necessary for cilial localization of the protein. Further, the first 15 amino acids of PC-2 are sufficient to localize heterologous proteins to cilia, such as cilia of kidney tubule epithelial cells, that do not otherwise traffic to cilia. In addition, assessment of PC-2 expression in cells lacking PC-1 showed that both native and epitope tagged over-expressed full length PC-2 are expressed normally in cilia. These findings support the presence of PC-2 in cilia of cells lining cysts of patients with mutations in PKD1 and use of channel agonists or antagonists targeted directly at PC-2 to alter the course of polycystic kidney disease in patients with mutations in PKD1.

The present invention relates to isolated peptides that localize to cilia, such as cilia of kidney tubule epithelial cells. Isolated PC-2 peptide is the subject of this invention. As described herein, the motif R₆VXP, which is at the NH2 terminus of PC-2, is necessary for cilial localization of PC-2.

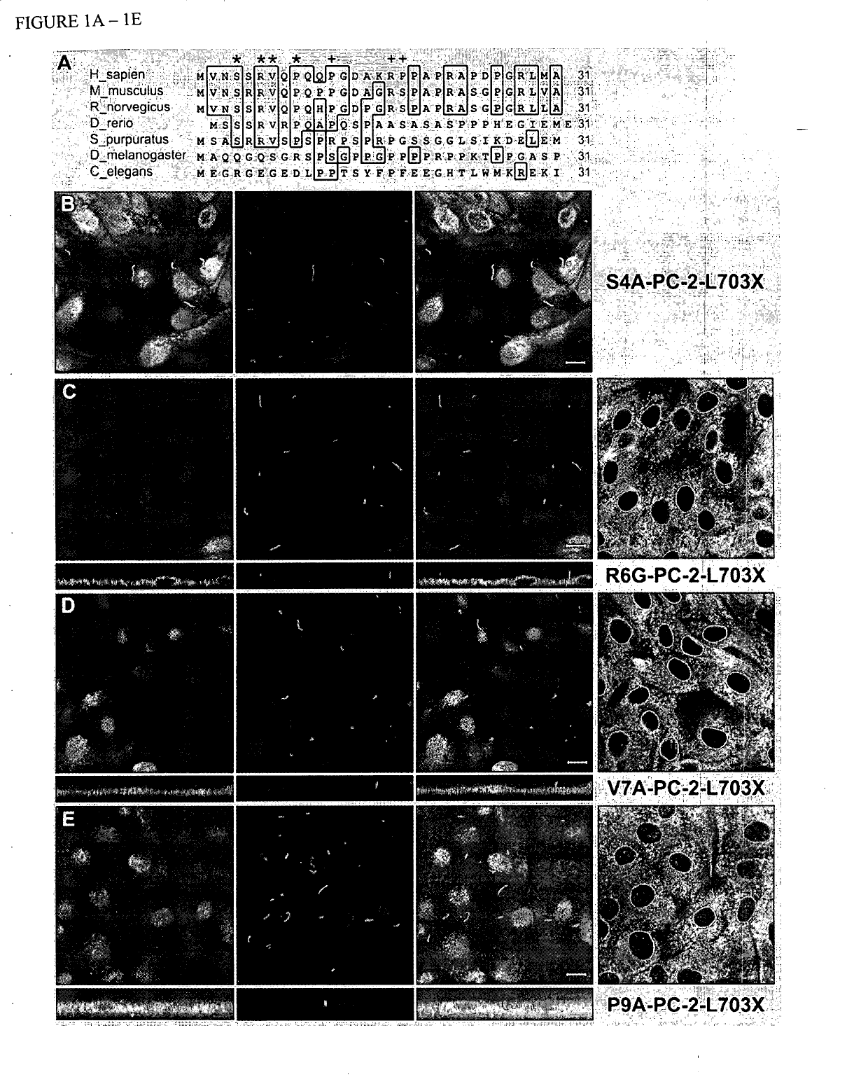

The present invention also relates to isolated peptides, referred to as trafficking peptides, that deliver to cilia, such as cilia of kidney tubule epithelial cell, heterologous proteins that do not normally (in the absence of a trafficking peptide) move to cilia. In one embodiment, trafficking peptides comprise the first 15 amino acid residues of polycystin-2, such human polycystin-2 or a functional equivalent of the first 15 amino acid residues of human PC-2. In this embodiment, the first 15 amino acid residues that comprise a trafficking peptide are those present in human PC-2: MVNSSRVQPQQPGDA (SEQ ID NO. 1). A functional equivalent of the trafficking peptide that comprises the first 15 amino acid residues of human PC-2 is a peptide in which at least one component amino acid residue is different from the amino acid residue in the corresponding position in the first 15 amino acid residues of human PC-2 and has substantially the same ability to deliver heterologous proteins to cilia as a targeting peptide that comprises the first 15 amino acid residues of human PC-2. Generally, a trafficking peptide functional equivalent comprises at least 3 amino acid residues that are identical to those present in the PC-2 peptide and will include the R₆VXP motif shown to be necessary for cilial localization of PC-2. In the motif, X can be, for example, Q, R, S, G or E, where Q represents glutamine; R represents arginine; S represents serine; G represents glycine and E represents glutamic acid. The first 31 amino acid residues of PC-2 from several species are shown in FIG. 1. The first 15 amino acid residues of each of the species represented in FIG. 1, as well as the corresponding amino acid residues of PC-2 of other species, can be assessed for their ability to traffic heterologous moieties, such as heterologous peptides, to cilia, such as cilia of kidney tubule epithelial cells, using methods described herein. In specific embodiments, the first 15 amino acid residues are the first 15 amino acid residues of H. sapien PC-2; M. musculus PC-2; R. norvegicus PC-2; D. rerio PC-2; or S. purpuratus PC-2.

A trafficking peptide can be used, for example, to deliver a heterologous protein or other moiety (e.g., a diagnostic agent, such as an imaging agent; a therapeutic agent) to cilia, such as cilia on kidney tubule epithelial cells. For example, it can be used to deliver or target therapeutic agents, such as calcium channel agonists (in specific embodiments, PC-2 agonists) to cilia in individuals (e.g., humans) who have ADPKD, in order to treat the condition and, as a result, prevent the progression of the condition or reverse the extent to which it exists. Alternatively, trafficking peptides of the invention can be used to deliver or target therapeutic agents to cilia in individuals at risk of developing ADPKD. As a result of this prophylactic use of trafficking peptides, ADPKD does not develop in individuals or develops to a lesser extent than would be the case if the trafficking peptides were not administered. The motif R₆VXP is necessary for cilial localization of PC-2. In specific embodiments, trafficking peptides comprise the motif R₆VXP, shown to be necessary for cilial localization of PC-2. In these embodiments, a peptide comprising the first 15 amino acid residues of human PC-2 or an equivalent peptide can be represented as: JJJJJRVXPJJJJJJ (SEQ ID NO. 2), in which each J represents an amino acid residue whose presence in the peptide results in or does not interfere with the peptide's cilial localization activity. Some or all of the constituent amino acid residues can be independently changed. For example, each amino acid residue represented by J can be that which occurs in the corresponding position in a PC-2 peptide represented in FIG. 1A or can be replaced by an amino acid other than that represented in FIG. 1A. There can be, for example, conserved amino acid substitutions.

In a further embodiment, the invention relates to a trafficking peptide in combination with, such as linked to, a heterologous moiety, such as a heterologous peptide, to be delivered to cilia, such as cilia of kidney tubule epithelial cells. The trafficking peptide and the heterologous moiety can be linked by, for example, a chemical bond or intervening amino acid residue(s), such as can be introduced when the trafficking peptide-heterologous protein are produced by recombinant DNA methods, chemical synthetic methods or peptide synthesis. In specific embodiments, the invention relates to trafficking peptide-heterologous moiety combinations in which the heterologous moiety is an imaging agent or a therapeutic agent. The trafficking peptide-heterologous moiety can be components of a singe entity, such as an entity in which they are fused/joined to one another. Such entities comprise at least one membrane span; the RVXP-containing sequence is cytosolic and in the N-terminus.

Therapeutic Compounds

In certain aspects, the present invention relates to PC-2 agonists for various therapeutic applications. As described herein, a “PC-2 agonist, also referred to as a PKD2 agonist” mimics or enhances PC-2 activities. PKD2 activities include, but are not limited to, a PC-2-mediated calcium signaling event such as PC-2-mediated calcium release in cells. For example, a PC-2 agonist may directly bind to a PC-2 protein or enhance interaction between PC-1 and PC-2 or bind to other proteins (e.g., syntaxin-5) that act to physiologically modulate the activity of PC-2. PC-2 agonists can be, for example, small organic molecules, proteins, antibodies, peptides, peptidomimetics, or nucleic acids.

In a specific embodiment of the present invention, a PC-2 agonist is a triptolide-related compound. As used herein, the term “triptolide-related compound” includes triptolide, triptolide prodrugs, and triptolide derivatives (e.g., analogs). Triptolide derivatives or prodrugs are capable of regulating calcium release in cells and/or bind to a calcium channel, such as PC-1 or PC-2. Calcium Modulation or regulation by triptolide-related compounds can be effected directly or indirectly; it is not necessary that a compound binds PC-1 or PC-2. With regard to structure, a triptolide “derivative” includes a compound derived from triptolide via a modification, which can include, for example: substitution of a hydrogen atom or hydroxyl group with hydroxyl, lower alkyl or alkenyl, lower acyl, lower alkoxy, lower alkyl amine, lower alkylthio, oxo (═O), or halogen; or conversion of a single bond to a double bond or to an epoxide. In this sense, “lower” preferably refers to C₁ to C₄, e.g., “lower alkyl” refers to methyl, ethyl, or linear or branched propyl or butyl. Preferred hydrogen atom substitutions include hydroxyl, methyl, acetyl (C(O)CH₃) and fluoro. A compound is considered to be derived from triptolide if it is produced from triptolide or synthesized with reference to or based on the chemical formula or structure of triptolide.

For example, triptolide-related compounds include triol-triptolide and triptonide. Other examples of triptolide derivatives and prodrugs include 14-methyl-triptolide, 14-deoxy-14α-fluoro-triptolide, 5α-hydroxy triptolide, 19-methyl triptolide, and 18-deoxo-19-dehydro-18-benzoyloxy-19-benzoyl triptolide, and 14-acetyl-5,6-didehydro triptolide, e.g., those described in U.S. Pat. Nos. 5,663,335, 5,962,516, 6,150,539, 6,458,537, 6,569,893, and 6,943,259 (each of these U.S. patents is hereby incorporated by reference in its entirety). The triptolide derivatives and prodrugs can be prepared from triptolide by methods such as those described therein.

In certain embodiments, any of the triptolide-related compounds having an ionizable group at physiological pH may be provided as a pharmaceutically acceptable salt. This term encompasses, for example, carboxylate salts having organic and inorganic cations, such as alkali and alkaline earth metal cations (for example, lithium, sodium, potassium, magnesium, barium and calcium); ammonium; or organic cations, for example, dibenzylammonium, benzylammonium, 2-hydroxyethylammonium, bis(2-hydroxyethyl)ammonium, phenylethylbenzylammonium, dibenzylethylenediammonium, and the like. Other suitable cations include the protonated forms of basic amino acids such as glycine, ornithine, histidine, phenylglycine, lysine, and arginine.

In certain embodiments, many of the triptolide-related compounds act as prodrugs, which are converted in vivo to triptolide. Compounds which are expected to convert to triptolide in vivo by known mechanisms, such as hydrolysis of an ester (organic or inorganic), carbonate or carbamate to an alcohol, or ring opening or ring closure from or to an epoxide or lactone, are referred to herein as prodrugs of triptolide or triptolide prodrugs. Such compounds are typically designed with such conversion in mind. These include, for example, the triptolide prodrugs described in U.S. Pat. Nos. 5,663,335, 5,962,516, 6,150,539, 6,458,537, and 6,569,893, and Published PCT Application No. WO 2003/101951. The present invention also relates to PC-2 agonists that can be obtained or identified from the screening methods described herein.

Drug Screening Assays

In certain embodiments, the present invention provides assays for identifying PC-2 agonists. Such PC-2 agonists may serve as therapeutic agents for various conditions, such as a cystic disease, cancer, or any condition caused by abnormal calcium signaling. In certain embodiments, agents of the invention specifically modulate PC-2 activities, for example, PC-2-mediated calcium release in cells. Optionally, a PC-2 agonist may directly bind to PC-2 or enhances interaction between PC-1 and PC-2. It is understood that PKD2 agonists include small organic molecules, proteins, antibodies, peptides, peptidomimetics, or nucleic acids.

In certain specific embodiments, the present invention is a screening assay that identifies agents that enhance PC-2-mediated calcium release in a test cell (e.g., a cell expressing PC-2). Alternatively, in an assay for agents that are specific to ADPKD, test cells that express PC-2 and do not express PC-1 can be used. In certain cases, the present invention relates to a screening assay that identifies PC-2 binding agents. The parameters detected in a screening assay may be compared to a suitable control. A suitable control may be an assay run previously, in parallel or later that omits the test agent. In an assay that is a suitable control, the same cell type (the control cell) as the test cell is subjected to/maintained under the same conditions (e.g., temperature, polarized state, confluent state, ciliated state) as those the test cell is subjected to/maintained under except for the agent(s) being assessed. Control cells and test cell are subjected to or maintained under the same conditions, except that control cells are not subjected to/maintained in the presence of the agent(s) being assessed. A suitable control may also be an average of previous measurements in the absence of the test agent. In general, the components of a screening assay mixture may be added in any order consistent with the overall activity to be assessed, but certain variations may be preferred.

In certain embodiments of the invention, assay formats include those which approximate such conditions as formation of ligand/receptor complexes, protein/protein complexes, PC-2-mediated calcium release, and anti-cyst activity. In certain cases, the assays may involve purified proteins or cell lysates, as well as cell-based assays which utilize intact cells. For example, simple binding assays can also be used to detect agents which bind to PC-2. Other binding assays may be used to identify agents that regulate interaction between PC-1 and PC-2.

In one embodiment of a binding assay, a test compound (a compound to be assessed for its ability to bind or otherwise interact with PC-2 protein) is contacted with a recombinant PC-2 protein. Detection and/or quantification of the test compound/PC-2 complex provides a means for determining the test compound's ability to bind to PC-2. In another embodiment of a binding assay, a test compound is contacted with a cell expressing PC-2. PC-2-mediated calcium release is measured in the cell in the presence of the test compound or in the absence of the test compound. If the test compound increases PC-2-mediated calcium release, the test compound is a PC-2 agonist. The efficacy of the compound can be assessed by generating dose response curves from data obtained using various concentrations of the test compound. A control assay can also be performed to provide a baseline for comparison. For example, in the control assay, the formation of complexes is detected and/or quantitated in the absence of the test compound. Another control is the use of cells that lack expression of PC-2; in the presence of test compound, there should be no calcium release response if there is no PC-2 expressed. Re-expression of PC-2 in these same cells should reconstitute the calcium response in the presence of the test compound. Such a control is useful, for example, since the fact that a compound binds PC-2 and releases calcium in cells, does not necessarily mean that it is releasing calcium through PC-2.

A further assay embodiment is one in which the focus is not on direct complexing of a test compound with PC-2. The focus is not on detecting binding of a test compound with PC-2, but, rather, on indirect interactions between a test compound and PC-2 that produce agonist effects in an individual. A compound identified through such an assay is an agonist that acts through a mechanism other than or in addition to direct binding to (physical complexing with) PC-2. Triptolide is an example of such an agonist.

In certain embodiments of the present invention, the test compounds in the screening assays can be any chemical (element, molecule, compound, drug), made synthetically, made by recombinant techniques or isolated from a natural source. For example, these compounds can be peptides, polypeptides, peptoids, sugars, hormones, or nucleic acid molecules (such as antisense or RNAi nucleic acid molecules). In addition, these compounds can be small molecules or molecules of greater complexity made by combinatorial chemistry, for example, and compiled into libraries. These libraries can comprise, for example, alcohols, alkyl halides, amines, amides, esters, aldehydes, ethers and other classes of organic compounds. These compounds can also be natural or genetically engineered products isolated from lysates or growth media of cells—bacterial, animal or plant—or can be the cell lysates or growth media themselves. Presentation of these compounds to a test system can be in either an isolated form or as mixtures of compounds, especially in initial screening steps.

In a further embodiment of the invention, a candidate agent (an agent to be assessed to determine if it is a PC-2 agonist) is identified as a PC-2 agonist in an animal model. In another further embodiment, a PC-2 agonist identified in such an in vivo model can be further characterized in an animal model for its therapeutic efficacy. The animal models include mice, rats, rabbits, and monkeys, which can be nontransgenic (e.g., wildtype) or transgenic animals. For example, the effect of the agent may be assessed in an animal model for any number of effects, such as its ability to slow or inhibit cyst growth in the animal and its general toxicity to the animal. Specific examples of such animal models include PC-1 or PC-2 deficient mice as described herein.

Pharmaceutical Compositions and Administration Methods

In certain embodiments of methods of the present invention, a PC-2 agonist is formulated with a pharmaceutically acceptable carrier. A PC-2 agonist can be administered alone or as a component of a pharmaceutical formulation. As described herein, the term “formulation” and “composition” are used interchangeably. A PC-2 agonist may be formulated for administration in any convenient way for use in human or veterinary medicine. In certain embodiments, a PC-2 agonist included in the pharmaceutical preparation may itself be active, or may be a prodrug. The term “prodrug” refers to compounds which, under physiological conditions, are converted into therapeutically active agents.

Formulations containing one or more PC-2 agonists (e.g., triptolide-related compounds) for use in the methods of the invention may take the form of solid, semi-solid, lyophilized powder, or liquid dosage forms, such as tablets, capsules, powders, sustained-release formulations, solutions, suspensions, emulsions, ointments, lotions, or aerosols, preferably in unit dosage forms suitable for simple administration of precise dosages. The compositions typically include a conventional pharmaceutical carrier or excipient and may additionally include other medicinal agents, carriers, or adjuvants.

In certain embodiments, the composition will be about 0.5% to 75% by weight of a compound or compounds of the invention, with the remainder consisting of suitable pharmaceutical excipients. In other embodiments, the composition will be more than 75% by weight of a compound or compounds of the invention. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Formulations of the PC-2 agonist include those suitable for oral, nasal, topical, parenteral, intravaginal and/or rectal administration. The formulations may be administered to a subject (individual) orally, transdermally or parenterally, e.g., by intravenous, subcutaneous, intraperitoneal, or intramuscular injection. The subject or individual is typically a human in need of therapy. For use in oral liquid preparation, the composition may be prepared as a solution, suspension, emulsion, or syrup, being supplied either in liquid form or a dried form suitable for hydration in water or normal saline. For parenteral administration, an injectable composition for parenteral administration will typically contain the PC-2 agonist in a suitable intravenous solution, such as sterile physiological salt solution. Liquid compositions can be prepared by dissolving or dispersing the PC-2 agonist (generally about 0.5% to about 20%) and optional pharmaceutical adjuvants in a pharmaceutically acceptable carrier, such as, for example, aqueous saline, aqueous dextrose, glycerol, or ethanol, to form a solution or suspension. Dosage forms for the topical or transdermal administration of the PKD2 agonist include powders, sprays, ointments, pastes, creams, lotions, gels, solutions, patches, and inhalants.

The PC-2 agonist may also be administered by inhalation, in the form of aerosol particles, either solid or liquid, preferably of respirable size. Such particles are sufficiently small to pass through the mouth and larynx upon inhalation and into the bronchi and alveoli of the lungs. In general, particles ranging from about 1 to 10 microns in size, and preferably less than about 5 microns in size, are respirable. Liquid compositions for inhalation comprise the active agent dispersed in an aqueous carrier, such as sterile pyrogen free saline solution or sterile pyrogen free water. If desired, the composition may be mixed with a propellant to assist in spraying the composition and forming an aerosol.

The formulations may conveniently be presented in unit dosage form and may be prepared by methods known to those of skill in the art. The amount of active ingredient which can be combined with a carrier material to produce a single dosage form will vary depending upon a variety of factors, such as the individual, such as a human, being treated and the particular mode of administration. The amount of active ingredient combined with a carrier material to produce a single dosage form will be, for example, an amount of the compound which produces a therapeutic effect.

Methods for preparing such dosage forms are known or will be apparent to those skilled in the art; for example, see Remington's Pharmaceutical Sciences (19th Ed., Williams & Wilkins, 1995). The composition to be administered will contain a quantity of the selected compound in an effective amount, for example for treating an ADPKD or ARPKD patient as described herein. To illustrate, for administration to human patients, a reasonable range of doses may be 0.1 to 20 mg, depending upon the activity of the derivative compared to that of triptolide. While i.v. administration is preferred in a clinical setting, other modes of administration, such as parenteral or oral, may also be used, with higher dosages typically used for oral administration.

Therapeutic Applications

In certain embodiments, the present invention relates to administration of a PC-2 agonist (e.g., a triptolide-related compound) for the treatment of a condition caused by abnormal calcium signaling, in slowing or inhibiting cyst growth, and in regulating calcium release (influx) and calcium signaling in cells. In one specific example, the present invention provides a method of treating or aiding in the treatment of polycystic kidney disease (e.g., ADPKD, ARPKD). Treatments include, but are not limited to, administration of a pharmaceutical composition, and may be prophylactic therapy, preventative therapy, or curative therapy (e.g., performed subsequent to the initiation of a pathologic event).

In one embodiment of the present invention, a PC-2 agonist (e.g., a triptolide-related compound) is administered prior to the development of symptomatic renal disease (e.g., cystic disease) in the individual for preventing PKD such as ADPKD. For example, the individual has been determined to be at risk of PKD as determined by family history, renal imaging study and/or genetic screening.

In a specific embodiment of the present invention, the PC-2 agonist (e.g., a triptolide-related compound) is administered when the individual exhibits symptomatic renal disease for preventing or treating PKD. As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. The term “treating” as used herein includes prophylaxis of the named condition or amelioration or elimination of the condition once it has been established.

There are many approaches to assessing effectiveness of treatment using methods and compositions of this invention. These include, but are not limited to reduced rate of cyst growth, reduced number of cyst, reduced rate of decline of kidney function (glomerular filtration rate, GFR), reduced occurrence of kidney failure requiring renal replacement therapy by dialysis modalities or kidney transplantation. Other possible outcomes can be reduced occurrence of high blood pressure in the PKD population, or reduced occurrence of intracranial aneurysms (which are associated with ADPKD in some families).

In certain embodiments, the present invention provides combination or multiple therapies for a condition such as PKD. For example, a PKD2 agonist (e.g., a triptolide-related compound) may be used in combination with other therapeutic agents. These additional therapeutic agents include, but are not limited to, antiviral agents, anticancer agents, and anti-inflammatory agents. In a specific embodiment, methods of the present invention comprises administering to an individual a therapeutically effective amount of a PKD2 agonist and a second therapeutic agent for treating PKD, such as an EGF receptor kinase inhibitor, a COX2 inhibitor, a vasopressin V₂ receptor inhibitor, a ligand of PTBR, a somatostatin analogue (e.g., octreotide), and pioglitazone. For example, triptolide, a precursor thereof (e.g., a prodrug) or a triptolide derivative can be administered to an individual in need of treatment, alone or in combination with each other (e.g., triptolide and a triptolide analogue) or with a second agent or drug (e.g., triptolide and an EGF receptor kinase inhibitor and/or others described herein). The second agent can be administered with a PC-2 agonist, either in the same formulation or in separate formulations, to enhance treatment. In these embodiments, the PC-2 agonist and the second agent can be administered at the same time (simultaneously) or at separate times (sequentially), provided that they are administered in such a manner and sufficiently close in time to have the desired effect.

In certain embodiments, methods of the present invention comprise administering a therapeutically effective amount of a PC-2 agonist. The phrase “therapeutically effective amount,” as used herein, refers to an amount which results in the decrease or inhibition of cell growth of target cells (e.g., those affected by abnormal calcium signaling). For example, a therapeutically effective amount of a PC-2 agonist slows or inhibits cyst growth.

The invention now being generally described, it will be more readily understood by reference to the following examples, which are included merely for purposes of illustration of certain aspects and embodiments of the present invention, and are not intended to limit the invention.

### EXEMPLIFICATION

Materials and Methods

The following materials and methods were used in the work described in Examples 1-6.

cDNA Expression Constructs

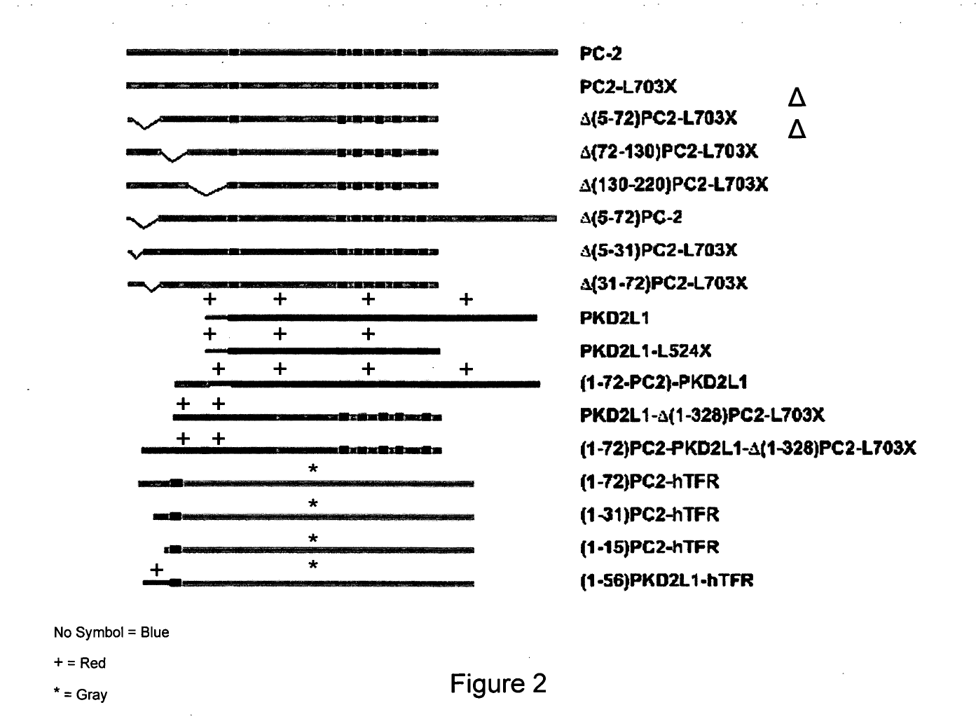

Schematics of the expression constructs used are presented in FIG. 2. All constructs were sequence verified. PC-2 and PC-2-L703X expression constructs in pcDNA3.1 (Invitrogen, Carlsbad, Calif.) have been previously reported (Cai et al., 1999). NH₂-terminal deletions took advantage of the presence of a unique SacII site in codon 72 of PKD2. Site directed mutagenesis was used to introduce a SacII site codon 5 to form Δ(delta). 5-72) deletion construct and in codon 130 to produce the Δ72-130) deletion constructs. For the Δ(130-220) deletion, Applicant first made a large deletion from codons 72 to 220 by introducing a SacII site at codon 220, followed by in-frame insertion codons 72-130 using the SacII sites generated above. RT-PCR amplification of a segment from the vector arm XbaI site upstream of codon 1 to codon 31 that included an engineered SacII site was used to replace codons 1-72 and form the Δ32-72 deletion construct. The Δ5-31 deletion was produced by inserting an RT-PCR product with codons 32-72 flanked by SacII sites into the Δ5-72 deleted peptide. Full length COOH-terminal EGFP tagged PC-2 was generated by cloning PKD2 in-frame into the pEGFP-N2 vector (Clontech, Mountain View, Calif.).

Applicant completed cloning of the full length PKD2L1 into pcDNA3.1/Zeo by RT-PCR from human brain cDNA using primers designed based on published sequences (Wu et al., 1998b; Nomura et al., 1998) and ligating the complete NH₂-terminus using the BamHI site in PKD1L1. A PKD2L1-EGFP fusion protein was constructed by inserting the complete PKD2L1 sequence in-frame into the pEGFP-N2 vector. RT-PCR from multiple tissues yielded an alternative form of the 5′ end of PKD1 L1) and, therefore, using site directed mutagenesis, Applicant generated a sequence to match that published by Nomura et. al. (Nomura et al., 1998).

Full length human transferrin receptor (hTFR) cDNA was kindly provided by P. De Camilli. Applicant PCR strategies were used to attach a 3×HA epitope tag to the COOH-terminus and clone it into pcDNA3.1. A SacII site was introduced by site directed mutagenesis at codon 61 of hTFR to allow replacement of the first 61 amino acids by various NH₂-terminal segments of PKD2 and PKD2L1.

Applicant took advantage of the unique BamHI site in the PKD2 and PKD2L1 cDNAs to swap the respective NH₂-terminal domains of each.

Point mutations were constructed using either the QuickChange site directed mutagenesis (Stratagene, La Jolla, Calif.) kit or conventional PCR.

Cell Culture and Transfection

LC-PK1 (CRL1392, ATCC) or MDCK (CRL6253, ATCC) cells were maintained in DMEM:F2 (1:1), 5% FBS, and penicillin/streptomycin at 37° C. in a 95% O₂/5% CO₂ incubator. Transfections were performed with Lipofectamine 2000 (Invitrogen, Carlsbad, Calif.) using 1 μg of plasmid DNA. Cells were selected 24 hours after transfection with 400 μg/ml G418 (Invitrogen, Carlsbad, Calif.) for 3 weeks. Positive colonies were verified by immunoblot and immunofluorescence analyses. Cells were grown on 0.4 μm pore size cell culture inserts (Becton Dickinson, Franklin Lakes, N.J.) for up to 21 days post confluence for analysis of cilia.

Immunofluorescent Cell Staining and Antibodies

For immunofluorescent labeling, cells were washed and fixed in freshly prepared 3.5% paraformaldehyde for 30 minutes followed by permeabilization with 0.1% Triton X-100. The cells were washed 3 times with PBS and then incubated with primary antibodies followed by subsequent incubation with the Alexa Fluor-conjugated secondary antibody (Molecular Probes, Eugene, Oreg.). Primary antibodies: acetylated α-tubulin monoclonal (6-11B-1Sigma, St. Louis, Mo.); polyclonal anti-HA epitope (71-5500, Zymed Laboratories, San Francisco, Calif.); rabbit polyclonal YCC2 (COOH-terminus) and YCB9 (NH₂-terminus) anti-PC-2 (Cai et al., 1999), mouse monoclonal anti-NaK-ATPase (clone 6H; kind gift from Michael Caplan), and mouse monoclonal anti-GM130 (kind gift from Graham Warren). Images were acquired using a Zeiss LSM 510 confocal microscope.

Immunoblot Analysis

Cell extracts were prepared by incubation in lysis buffer (10 mM Tris pH 7.0, 1 mM EDTA, 1 mM PMSF, 10 μg/ml leupeptin, 10 μg/ml pepstatin, 1 μg/ml aprotinin) for 30 minutes at 4° C. Lysate was centrifuged 8,000×g for 15 minutes and dissolved in Laemmli SDS buffer followed by incubation at 65° C. for 5 minutes. Proteins were separated by SDS-PAGE (4-15%) and electrophoretically transferred to PVDF membranes overnight at 20 mV in Tris-glycine-methanol. After blocking 1 hour (5% dried milk), membranes washed three times with TBS-TApplicanten (0.05%), incubated for 1 hour with the primary antibody, washed, incubated 1 hour with horseradish peroxidase-coupled secondary antibody, washed extensively and processed for chemiluminescence by ECL (Amersham, Arlington Heights, Ill.).

Glycosylation and Cell Surface Biotinylation Analysis

Cell lysate and membrane fraction protein were treated with peptide:N-glycosidase F (PNGase F) or endoglycosidase H (Endo H) (New England Biolabs, Beverly, Mass.) and analyzed by SDS-PAGE followed by immunoblotting as described previously (Cai et al., 1999; Koulen et al., 2002). Cell surface proteins were biotinylated as described by Gottardi et. al. (Gottardi et al., 1995). Solutions were pre-chilled and all incubations performed at 4° C. Cells grown as described above were washed once in serum free DMEM and twice with PBS-CM (PBS supplemented with 0.1 mM CaCl₂, 1.0 mM MgCl₂) followed by two incubations of 25 minutes each with biotin solution (10 mM triethanolamine pH 9.0, 150 mM NaCl, 2 mM CaCl₂, and 0.5 mg/ml sulfo-NHS-LC-biotin [Pierce, Rockford, Ill.]). The biotin reaction was quenched with PBS-CM and 100 mM glycine for 20 minutes. Cells were rinsed twice with PBS-CM and incubated in 1 ml lysis buffer (50 mM Tris pH 7.4, 150 mM NaCl, and 1% Triton X-100) for 60 minutes. Lysates were centrifuged at 14,000×g for 10 minutes and 800 □l of the cleared supernatant was immunoprecipitated with 30 μl of agarose conjugated streptavidin overnight at 4° C. The beads were washed three times with lysis buffer, once with high salt buffer (lysis buffer supplemented with 500 mM NaCl and 0.1% Triton X-100) and once with no-salt buffer (10 mM Tris, pH 7.4). Beads were re-suspended in protein sample buffer and subjected to SDS-PAGE and immunoblotting.

Live Cell Immunofluorescent Labeling

Solutions were pre-chilled and all incubations performed at 4° C. Cells grown on cell culture inserts were washed twice with surface buffer: PBS supplemented with 0.1 mM CaCl₂, 1.0 mM MgCl₂ and incubated with anti-HA antibody on both apical and basolateral surfaces, followed by three washes and incubation with the Alexa Fluor-conjugated antibody in surface buffer. Cells were washed and fixed for immunofluorescence as described above.

Cytosolic Ca²⁺ Measurements

Cells were plated on glass coverslips and used within 16-48 hours. The coverslips were rinsed in HBSS (19.7 mM HEPES pH 7.4, 130 mM NaCl, 5 mM KCl, 1.2 mM KH₂PO₄, 1 mM MgCl₂, and 1.25 mM CaCl₂) and incubated with fluo-4AM (10 μM in HBSS) for 30 minutes at 37° C. The coverslips were rinsed in HBSS, mounted in a perfusion chamber, and analyzed with a Ziess Aviovert S100 confocal microscope. Fluo-4 was excited at 488 nm and time-lapse emission recorded at 522 nm. Fluorescence amplitudes (F) were normalized to baseline (F₀) and reported as F/F₀. The t_{1/2} value corresponds to the time required for the fluorescence to decrease to one-half its peak amplitude.

### Example 1

### PC-2 Trafficking to Cilia is Independent of PC-1

Full length PC-2 is retained in the ER in cultured epithelial cells in the non-ciliated state. Once cells form cilia, in addition to its ER location, full length epitope-tagged PC-2 traffics to the plasma membrane overlying primary cilia in both LLC-PK1 and MDCK epithelial cells. PC-2 is not detected on the remainder of the cell surface plasma membrane (Cai et al., 1999; Koulen et al., 2002; Cai et al., 2004). Truncation of the COOH-terminus of PC-2 upstream of amino acid E787 results in trafficking of the residual protein to the general cell surface even before cilia form (Cai et al., 1999; Koulen et al., 2002). Applicant determined that addition of a COOH-terminal EGFP fusion to full length PC-2 does not alter cilial trafficking properties of the full length protein. The truncation mutant, PC-2-L703X, lacking the bulk of the COOH-terminus including the putative PC-1 interaction domain (Qian et al., 1997; Tsiokas et al., 1997) also shows robust expression in cilia in either LLC-PK1 or MDCK cells (FIG. 1B). Previous studies had suggested that co-assembly with PC-1 is required for surface expression of PC-2 (Hanaoka et al., 2000) and that PC-2 is not expressed in cilia in the absence of PC-1 (Nauli et al., 2003). The specific trafficking of full length PC-2 to cilia and the ability of the truncated PC-2-L703X form to also achieve this localization led us to investigate whether full length PC-2 can traffic to cilia independently of PC-1.

To test whether PC-1 is necessary for localization of PC-2 to cilia, Applicant examined PC-2 trafficking in the absence of PC-1 in several experimental systems. Results showed that native PC-2 is expressed in nodal cilia of Pkd1^{-/-} mice, indicating that in nodal cilia, PC-2 traffics to cilia independently of PC-1. Similarly, in cells lining kidney cysts formed by inactivation of Pkd1 (S. Shibazaki, X. Tian, S. Somlo), cilia express PC-2 in a pattern indistinguishable from that seen in non-cystic tubule cells. PC-2 is also expressed in cilia of Pkd1 null cell lines constructed from mice. (Chauvet et al., 2004). Finally, Applicant expressed full length PC-2 as a COOH-terminal EGFP fusion protein in Pkd1^{-/-} cells and found EGFP epifluorescence in the cilia (FIG. 2 D). The data indicate that PC-2 localizes in cilia in the absence of PC-1.

### Example 2

### An NH₂-Terminal Domain Necessary for Cilial Location of PC-2

Applicant sought to define the domains of PC-2 that direct it specific trafficking properties. For this purpose, Applicant produced a series of deletion and fusion constructs of human PC-2. Deletion of amino acids 5-72 resulted in loss of cilial location of the COOH-terminal truncated peptide Δ(5-72)PC2-L703X. By contrast, deletion of amino acids 72-130 or 130-220 had no effect on trafficking to cilia of the respective Δ(72-130)PC2-L703X and Δ130-220)PC2-L703X peptides. Applicant excluded the possibility that loss of cilial location resulted from differences in the expression of the mutant peptides. Robust expression of the proteins deleted for amino acids 5-72 was seen in cell body using confocal microscopy and the proteins showed the expected expression and migration by SDS-PAGE of total cell lysates detected by immunoblotting anti-HA epitope antibodies.

Since the PC-2-L703X backbone lacks the COOH-terminus of PC-2, Applicant considered the possibility that the intact COOH-terminus of PC-2 may override the role of the NH₂-terminus in localizing full length PC-2 to cilia. In contrast to full length PC-2, the Δ(5-72)PC-2 construct deleted for amino acids 5-72 but containing the intact COOH-terminus failed to traffic into cilia. The finding that (Δ5-72)PC-2 protein does not traffic into cilia, indicates that NH₂-terminal sequences are essential for cilial location and that the capacity to interact with PC-1 via the COOH-terminus is not sufficient to deliver PC-2 to cilia.

### Example 3

### Subcellular trafficking of NH₂-terminal domain mutants of PC-2

Applicant examined the differences in the intracellular trafficking of PC-2-L703X and Δ(5-72) PC2-L703X. The proteins both showed a perinuclear and cytosolic expression pattern and co-localization with calnexin (data not shown) indicative of location in the ER. PC-2-L703X also showed partial co-localization with the Golgi marker GM130 (Nakamura et al., 1995). The Δ(5-72) PC2-L703X form did not co-localize with this Golgi marker, indicating that deletion of the NH₂-terminal domain of PC-2 results in failure to traffic into the Golgi.

Glycosylation analysis in ciliated cells was used to further examine trafficking of these proteins. Normally, the fraction of PC2-L703X that is expressed on the cell surface acquires resistance to Endoglycosidase H (Endo H) as it passes through the middle Golgi on its way to the plasma membrane. The Endo H resistant species appears as a slower migrating band in immunoblots (Cai et al., 1999; Cai et al., 2004). It is important to emphasize that forms of PC-2 with the COOH-terminus deleted that traffic to cilia (e.g., PC2-L70X, Δ(72-130) PC2-L703X and Δ(130-220) PC2-L703X) also traffic to the general plasma membrane outside the cilia and do so even in the absence of cilia formation [see below and (Cai et al., 1999; Cai et al., 2004)]. By contrast, PC-2 with an intact COOH-terminus only expressed on the cell surface once cells form cilia, and then is only found in the plasma membrane overlying the cilia and not expressed in the remainder of the cell surface. Therefore, there are far greater amounts of the truncated forms of PC-2 on the cell surface than of the intact protein, even in ciliated cells (see below). Since passage through the middle Golgi is required to develop Endo H resistance, mutant forms of PC-2 that reach the cell surface are expected to acquire Endo H resistance. The Δ(5-72) PC2-L703X form of PC-2 is expected to remain completely sensitive to Endo H digestion since it does not traffic into the Golgi nor reach the cell surface. Results showed that PC2-L703X, Δ(72-130) PC2-L703X and Δ(130-220) PC2-L703X acquire Endo H resistance. By contrast, the NH₂-terminal deleted Δ(5-72) PC2-L703X protein did not acquire Endo H resistance. This is consistent with the observed failure of Δ(5-72) PC2-L703X to reach the Golgi compartment and the cilia and supports the conclusion that deletion of amino acids 5-72 of PC-2 results in retention of the protein in pre-Golgi compartments.

Surface biotinylation in living cells was used to assess cell surface expression of PC-2 in ciliated cells. Previous work has shown absence of detectable surface-biotinylated PC-2 in non-ciliated cells (Cai et al., 1999; Cai et al., 2004). Assessment was carried out to determine if surface-biotinylated PC-2 is detectable in ciliated cells. Full length PC-2 with an epitope tag traffics to cilia (Cai et al., 2004). However, Applicant was unable to detect biotinylated full length PC-2 on either the apical or basolateral surface of living, polarized, ciliated epithelial cells. Full length PC-2 also does not have detectable Endo H resistant forms (Cai et al., 1999; Koulen et al., 2002; Cai et al., 2004). Applicant concluded that cell surface biotinylation and Endo H resistance do not have sufficient sensitivity to detect glycoproteins such as PC-2 whose surface expression is restricted only to cilia. The most likely interpretation of our data is that surface expression of full length PC-2 is restricted to the plasma membrane compartment overlying cilia in ciliated cells and that PC-2 does not otherwise traffic to the general plasma membrane in epithelial cells.

Removal of the COOH-terminus allows PC2-L703X protein to traffic to the general plasma membrane, as well as to the cilia. As a result, PC2-L703X is biotinylated on both the apical and basolateral surfaces in polarized, ciliated cells (Cai et al., 1999; Cai et al., 2004). Only the slower migrating, Endo H resistant form of PC2-L703X is biotinylated, confirming the interpretation that the Endo H resistant form of PC2-L703X represents the fraction of the protein on the cell surface. Despite absence of the COOH-terminus, no detectable biotinylated form of Δ(5-72) PC2-L703X is seen in ciliated polarized cells. In contrast to full length PC-2, the absence of detectable surface biotinylation and Endo H resistance in Δ(5-72) PC2-L703X is coupled with absence of detectable immunofluorescent signal in cilia. Taken together, this shows that Δ(5-72)PC2-L703X is not expressed on the plasma membrane at all, even when cells form cilia. Applicant concluded that amino acids 5-72 are necessary for trafficking of PC-2 to the plasma membrane and cilial compartments.

### Example 4

### The PC-2 Homolog, PKD2L2, is not Expressed in Cilia in Epithelial Cells

Applicant evaluated whether cilia location is a general property of polycystin class of TRP channels (TRPP). PC-2 and PKD2L1 have ˜60% identity and 80% similarity over the region between the first and sixth membrane spans (the TRP channel region) and have 40% identity and 60% similarity in the first 200 amino acids of their cytosolic COOH-termini. PC-2 and PKD2L1 have no similarity in the NH₂-terminus and diverge significantly in the remainder of their respective COOH-termini. Applicant reasoned that these proteins are similar in structure and both function as Ca²⁺ channels. They sought to examine the specificity of the NH₂-terminal trafficking domain of PC-2 by comparing it with the trafficking of PKD2L1.

Applicant completed the cloning of the 5′ end of a partial PKD2L1 sequence (Wu et al., 1998b) and identified a novel splicing pattern from exon 1 to exon 2. This splice occurred at a site 22 base pairs upstream of the exon 2 splice acceptor site reported by Nomura et. al. (Nomura et al., 1998). The novel variant extended exon 2 in the 5′ direction and changed the predicted in-frame translation start site in exon 1. As a result, the first 78 amino acids of the previously reported PKD2L1 sequence (GenBank Accession NM016112) are replaced by a novel 31 amino acid sequence (GenBank Accession DQ084244); the remainder of the protein is unchanged. Using direct sequencing of RT-PCR products formed with closely spaced PCR primers flanking the exon 1-2 splice site, Applicant did not find evidence for the previously reported splicing pattern between exons 1 and 2. Unless otherwise noted, PKD2L1 expression constructs with the newly defined NH₂-terminal sequence were used in work described herein.

PKD2L1 with either EGFP or triple-HA COOH-terminal epitope tags was only detected in the cytoplasm and perinuclear areas of LLC-PK1 cells stably expressing the proteins. PKD2L1 does not traffic to cilia in polarized, ciliated LLC-PK1 cells. Applicant also engineered the PKD2L1 with the NH₂-terminal sequence described by Nomura et. al. (Nomura et al., 1998) and found that this form of PKD2L1-EGFP does not traffic to cilia. PKD2L1 does not acquire Endo H resistance, consistent with the finding that it does not traffic to the cell surface in epithelial cells. Cell surface biotinylation in living LLC-PK1 and CHO-K1 cells similarly found no evidence of PKD2L1 on the cell surface in either cell type. Finally, Applicant constructed a truncation of PKD2L1, PKD2L1-L524X (FIG. 2), removing the predicted cytosolic COOH-terminus to produce a protein analogous to PC2-L703X. PKD2L1-L524X was also not biotinylated on the cell surface and did not have a cell surface pattern of immunofluorescent cell staining. In aggregate, these data indicate that PKD2L1 does not contain plasma membrane or cilia targeting information in its primary sequence and does not normally traffic to the plasma membrane.

Applicant evaluated the channel function of PKD2L1 in LLC-PK1 cells. Since this protein was not expressed on the cell surface, Applicant assayed its ability to release Ca²⁺ from intracellular stores in the absence of extracellular Ca²⁺ in a manner similar to that of PC-2 (Koulen et al., 2002). PKD2L1 was able to transiently increase cytosolic Ca²⁺ in response to AVP stimulation in a manner similar to PC-2, suggesting PKD2L1 can function as a Ca²⁺ channel in this assay system (Koulen et al., 2002; Chen et al., 1999; Rundle et al., 2004).

### Example 5

### The NH₂-Terminal Domain of PC-2 is Sufficient to Direct Trafficking to Cilia

Applicant tested whether the NH₂-terminal 72 amino acids of PC-2 are sufficient to direct to cilia proteins that do not normally traffic to cilia. The initiation codon of PKD2L1 was replaced with codons 1-72 of PC-2 resulting from the chimeric protein, (1-72-PC2)-PKD2L1, that contained the first 72 residues of PC-2 and the entire PKD2L1 sequence (FIG. 2). This chimeric protein trafficked into cilia of epithelial cells. To further confirm that the NH₂-terminus of PKD2L1 did not contain a latent cilia targeting motif, Applicant produced a chimeric protein in which Applicant replaced amino acids 1-328 of PC2-L703X were replaced with the corresponding region (amino acids 1-230) of PKD2L1 [PKD2L1-Δ(1-328) PC2-L703X, FIG. 2]. The chimeric region contained the entire cytosolic NH₂-terminus, the first transmembrane span and part of the first extracellular loop of PKD2L1, replacing the comparable region of PC2-L703X. This chimeric protein failed to traffic to cilia. When amino acids 1-72 of PC-2 were added back to the NH₂-terminus of PKD2L1-Δ(1-328) PC2-L703X (FIG. 2), the doubly chimeric protein localized to cilia.

Applicant developed a heterologous trafficking assay using the human transferrin receptor (hTFR) to test whether this NH₂-terminal domain of PC-2 was effective in targeting other type II membrane proteins to cilia. hTFR has a 67 amino acid NH₂-terminal cytoplasmic tail containing a Y₂₀XRF internalization motif (Jing et al., 1990; Collawn et al., 1990) followed by a single membrane span and a 671 amino acid extracellular COOH-terminal domain. A triple HA-epitope was placed at the extracellular COOH-terminus of hTFR. Assessment confirmed that the full length protein does not traffic into the cilia. Amino acids 1-61 in the cytosolic NH₂-terminus of hTFR were replaced with amino acids 1-56 from the NH₂-terminus of PKD2L1. This chimeric protein was confined to intracellular membrane compartments and failed to traffic to cilia. When Applicant replaced the first 61 amino acids of hTFR with the first 72 amino acids of PC-2, the chimeric protein trafficked selectively to the apical membrane and showed robust localization in the cilial membrane of these cells. A doubly chimeric protein in which the first 56 amino acids of PKD2L1 preceded the first 72 amino acids of PC-2 fused with hTFR did not traffic to cilia (data not shown). This suggests that accessibility of the PC-2 trafficking domain at the NH₂-terminus is important to its function. These data strongly suggested that first 72 residues of the NH₂-terminus of PC-2 contained a domain sufficient for directing targeting to cilia.

### Example 6

### An RVXP Motif is Required for Trafficking of PC-2 to Cilia

Applicant next sought to better define this domain and identify the responsible motif. L703X-HA deleted for amino acids 31-72 [Δ(31-72)PC2-L703X] still trafficked to cilia whereas deletion of amino acids 5-31 [Δ(5-31)PC2-L703X] abrogated cilial trafficking. Applicant checked the trafficking to cilia of hTFR chimeras containing the first 31 and first 15 amino acids of PC-2 and found both localized to cilia. The apical and cilial location of the fusion of the first 15 amino acids of PC-2 with hTFR was confirmed by labeling the outside HA epitope in living, non-permeabilized cells. The first 15 amino acids of PC-2 define a minimal domain sufficient for apical membrane and cilia location.

Applicant compared sequences of the first 31 amino acids of PC-2 from several species and found that the highest degree of conservation occurred within the first 15 amino acids (FIG. 1A). A potentially conserved motif S—X—R—V—X—P occurred in vertebrate forms of PC-2 as well as sea urchin (FIG. 1A). This motif is not conserved in PKD2L1. Applicant introduced the following point mutations into PC2-L703X backbone: S4A, R6G, V7A, and P9A within this putative motif and P12A, R17G and P18A in non-conserved residues (FIG. 1A). Mutations at residues S4, P12, R17 and P18 had no effect on cilia location of PC2-L703X (FIG. 1 B and data not shown). Mutations R6G, V7A, and P9A each resulted in loss of cilia location of PC2-L703X (FIGS. 1C, D, E). These data suggest that the residues in the motif RVXP are necessary for location of PC-2 in cilia.

### DISCUSSION

The primary cilium has taken center stage in a broad class of human diseases with pleiotropic manifestations that often include cyst formation and fibrosis in the kidney and liver (Pazour, 2004; Zhang et al., 2004). Of these diseases, autosomal dominant polycystic kidney disease (ADPKD) is the most common and invariably manifests with cysts in the kidney. The two gene products associated with ADPKD, polycystin-1 (PC-1) and polycystin-2 (PC-2), are integral membrane proteins that localize in the plasma membrane overlying the cilial axoneme of kidney tubule epithelial cells (Pazour et al., 2002; Yoder et al., 2002a). PC-2 is also abundantly expressed in the ER where it can function as a calcium release channel (Koulen et al, 2002). The relative contributions of PC-2 function in the cilia and the ER to the ADPKD phenotype is unknown, although it is reasonable to hypothesize that its role in the cilium is essential. The single cilium on the luminal (apical) surface of these cells functions as a sensory organelle (Pan et al., 2005) and PC-1 and PC-2 form part of the signal transduction pathway in this structure. The precise nature of the extracellular signal is uncertain, but a role for mechanical fluid shear stress has been proposed (Praetorius and Spring, 2001; Nauli et al., 2003). PC-1 has structural features of a cell surface receptor and undergoes proteolytic cleavage that is required for its function (Qian et al., 2002; Chauvet et al., 2004). PC-2 is a cation channel belonging to the TRP channel family (Mochizuki et al., 1996; Gonzalez-Perrett et al., 2001; Koulen et al., 2002). PC-1 physically associates with PC-2 (Qian et al., 1997; Tsiokas et al., 1997; Hanaoka et al., 2000) and Ca²⁺ is involved in the intracellular signaling steps from this complex (Nauli et al., 2003; McGrath et al., 2003).

Although inherited as a dominant trait, cyst formation in ADPKD occurs after somatic second step mutations (Qian et al., 1996; Wu et al., 1998a). Cyst cells lack functional forms of either PC-1 or PC-2, depending on the genotype of the affected family. Several lines of evidence support the functional inter-relationship of PC-1 and PC-2 in the kidney, liver and pancreas. The spectrum of clinical features of human ADPKD is the same for PKD1 and PKD2, except that the former is more severe at any given age (Hateboer et al., 1999). Pkd1^{-/-} and Pkd2^{-/-} mice, as well as the respective heterozygous mice, have very similar kidney, liver and pancreas phenotypes (Lu et al., 1997; Lu et al., 2001; Wu et al., 2000; Wu et al., 2002; Kim et al., 2000). Mutations in the orthologous genes in C. elegans, lov-1 and pkd-2, result in male mating defects that are identical to each other (Barr et al., 2001). On the other hand, compound heterozygous Pkd1^{+/-}:Pkd2^{+/-} mice differ from singly heterozygous animals by a mild extra-additive effect in cyst formation (Wu et al., 2002), raising the possibility of a partial epistatic relationship between these genes. Outside of the kidney, liver and pancreas, there is more striking evidence of functional divergence for PC-1 and PC-2. Pkd2^{-/-} mice develop defects of left-right axis formation with associated heart defects reflective of heterotaxy syndrome (Pennekamp et al., 2002; Wu et al., 2000). This results from failure to express functional PC-2 in nodal cilia (McGrath et al., 2003) and is not seen in Pkd1^{-/-} mice (unpublished observations), separating PC-2 from PC-1 in the nodal cilial pathway.

Under non-ciliated conditions, PC-2 expression is confined to the endoplasmic reticulum (Cai et al., 1999) where it can release Ca²⁺ from intracellular stores (Koulen et al., 2002). Truncation of the cytosolic COOH-terminus allows trafficking of the peptide to plasma membrane where it acquires resistance to Endoglycosidase H (Endo H) and can be biotinylated in living cells (Cai et al., 1999; Cai et al., 2004). Heterologously expressed, epitope tagged PC-2 traffics into cilia once they form in post-confluent monolayers but the full length epitope tagged protein still cannot be detected on the remainder of the plasma membrane (Cai et al., 2004). Although cell surface location of endogenous PC-2 has been reported using a variety of native protein antisera (Luo et al., 2003; Scheffers et al., 2004; Li et al., 2005), none of these studies showed expression of epitope tagged PC-2 in the plasma membrane. This has led to some controversy regarding PC-2 expression in the plasma membrane outside cilia [reviewed in (Witzgall, 2005)]. The domain responsible for restricting PC-2 to the ER and cilia is a stretch of acid residues containing a casein kinase 2 phosphorylation site at S812 (Cai et al., 1999; Cai et al., 2004; Kottgen et al., 2005). Loss of phosphorylation at S812 does not abrogate cilial localization of PC-2 (Cai et al., 2004). An interdependence with PC-1 for PC-2 trafficking to the cell surface was inferred from the presence of a novel channel conductance in cells over-expressing both proteins, although the identity of that conductance with the PC-2 channel was never demonstrated (Hanaoka et al., 2000). More recently, native PC-2 was deemed to be absent from cilia of cultured epithelial cells lacking functional PC-1 (Nauli et al., 2003).

There is now general agreement supporting the functional role of PC-1 and PC-2 in cilia. Many of the trafficking studies for both proteins were performed before the role of cilia was widely appreciated. Applicant sought to examine the mechanism for cilia trafficking of PC-2 with the view that this is central to pathogenesis of polycystic diseases. Specifically Applicant set out to test the hypothesis that PC-2 has endogenous cilial trafficking information within its primary sequence and that this trafficking is independent of PC-1. Applicant based these hypotheses on the observation that COOH-terminal truncated forms of PC-2 that do not interact with PC-1 still traffic to cilia. As described herein, Applicant found the motif, R₆VXP, at the NH₂-terminus of PC-2 is necessary for cilial location of the protein. Applicant also found that the first 15 amino acids of PC-2 are sufficient to localize heterologous proteins to cilia that do not otherwise traffic to cilia. Finally, Applicant examined PC-2 expression in cells lacking PC-1 and found that both native and epitope tagged over-expressed full length PC-2 are expressed normally in cilia.

Cilia play a central role in a spectrum of human diseases that include autosomal dominant and recessive polycystic kidney disease, nephronophthisis and Bardet-Biedl syndrome (PazOur, 2004; Pan et al., 2005). The proteins encoded by genes mutated in these diseases localize to the cilia complex that includes the basal body, the cilial axoneme and the overlying plasma membrane. From an alternative perspective, induced mutations in genes encoding several intraflagellar transport (IFT) proteins not known to be involved in human disease nonetheless result in polycystic phenotypes in model organisms (PazOur et al., 2000; Yoder et al., 2002b; Lin et al., 2003; Sun et al., 2004). It follows that polycystic kidney disease can result from defective ciliogenesis or from loss of discrete cilial proteins in otherwise structurally normal appearing cilia. Human ADPKD falls into the latter category (Thomson et al., 2003). Understanding how the ADPKD proteins get to cilia is essential to understanding both the function of the respective disease gene products and the role of cilia in the disease.

Cilial Targeting of PC-2

The cilium is a privileged compartment with restricted access from the contiguous cytosolic and membrane compartments (Rosenbaum and Witman, 2002). Vesicles containing integral membrane proteins destined for cilia travel via to the area of the base of the cilium (Bouck, 1971) where there is a structural delimitation of the cilial and extra-cilial domains (Satir et al., 1976), perhaps corresponding to the transition zone (O'Toole et al., 2003). It has been proposed that there are cilial targeting signals for directing membrane and axonemal proteins to the cilial compartment (Rosenbaum and Witman, 2002). A protein may traffic to cilia either by having such a signal in its primary sequence or by forming a pre-assembly complex in the cytoplasm with another protein(s) that has such a signal. The compartmental restriction between the cilium and the remainder of the cell (Nir et al., 1984) is mediated via a ‘flagellar pore complex’ comprised of transition fibers extending from the distal portion of the basal body to the plasma membrane (Deane et al., 2001). It is generally accepted that vesicles do not traffic into cilia (Kozminski et al., 1993). Instead, cilial integral membrane proteins like the polycystins are synthesized in the ER and trafficked through the Golgi into post-Golgi vesicles that dock near the cilial pore complex between the basal body and cilium (Bouck, 1971; Rosenbaum and Witman, 2002). A plausible alternative mechanism would require that vesicles fuse with the apical membrane compartment adjacent to the cilium and from where membrane proteins enter the cilial compartment via an intramembranous process (Witzgall, 2005).

Distinct compartmentalization of the cilial and apical plasma membranes is illustrated by PC-2, which is concentrated in the plasma membrane overlying the cilial axoneme and is not detected on the remainder of the cell surface (Cai et al., 1999; Cai et al., 2004) and gp135 (podocalyxin) which marks the apical membrane but is excluded from cilia (Meder et al., 2005). The restricted distribution of PC-2 requires a balance of two processes—one that is permissive for entry into the cilial compartment and the other that is either restrictive of exit from, or exclusive of entry into, the contiguous apical plasma membrane compartment. The presence of wild type PC-2 only in the cilial plasma membrane suggests that both permissive and restrictive (or exclusive) processes are active. They are likely mediated by distinct domains. The distribution of the PC2-L703X truncation to both the cilial and non-cilial plasma membrane suggests that PC2-L703X retains the signals permissive of entry to the cilial membrane but has lost signals restricting it only to that compartment.

Applicant found a signal permitting access to the cilial plasma membrane resides within the primary sequence of the NH₂-terminus of PC-2 in an RVXP motif that is conserved among vertebrate forms of the protein. Previous to this, fOur flagellar/cilial targeting domains have been reported (Nasser and Landfear, 2004; Ersfeld and Gull, 2001; Godsel and Engman, 1999; Tull et al., 2004). All are in species of the protozoa Leishmania and Trypanosoma and only one of these is in a flagellar membrane protein (Nasser and Landfear, 2004). The ISO1 glucose transporter in Leishmania enriettii contains the necessary targeting information in at least two segments of its cytosolic NH₂-terminus (Nasser and Landfear, 2004). The other three reported domains are in flagellar axonemal proteins. In Trypanosoma brucei, the paraflagellar rod protein A (PFRA) and an actin-related protein (TrypARP) share a COOH-terminal histidine-leucin-alanine (HLA) motif that is necessary for targeting to the flagellum (Ersfeld and Gull, 2001). The flagellar Ca²⁺-binding protein (FCaBP) of Trypanosoma cruzi as well as the SMP-1 protein of Leishmania major require NH₂-terminal myristoylation and palmitoylation on respective glycine and cysteine residues to achieve flagellar location (Godsel and Engman, 1999; Tull et al., 2004). The motif in FCaBP is the only one previously shown to be both necessary and sufficient for flagellar location (Godsel and Engman, 1999).

The RVXP motif is likely part of a protein interaction domain. It is not likely to be a lipid interaction domain since it does not have an NH₂-terminal G or a C typical of the myristoylation and palmitoylation sites, respectively, as observed for axonemal proteins in unicellular organisms (Godsel and Engman, 1999; Tull et al., 2004). The highly selective trafficking to the apical membrane and cilia of the (1-15) PC2-hTFR chimeric protein suggests that this motif directs trafficking of post-Golgi vesicles to either the cilial pore complex or the apical membrane. The trafficking of the polytopic PC2-L703X is more complex since it also detectable on the basolateral membrane as well. Once cells polarize and form cilia, PC2-L703X appears to preferentially accumulate in the apical membrane compartment. The only NH₂-terminal binding partner for PC-2 described to date is α-actinin (Li et al., 2005). It is associated with the actin rather than microtubule cytoskeleton, is not known to be localized in cilia and has been proposed to function in modulating the channel activity of PC-2 (Li et al., 2005). It is an unlikely candidate for a role in trafficking of PC-2 into cilia. PC-2 has been found to interact with intraflagellar transport proteins (IFT57) and pericentrin, a protein that is necessary for proper formation of cilia (Jurczyk et al., 2004). The association with pericentrin occurs at the level of the centriole/basal body (Jurczyk et al., 2004) while the association with a putative trafficking partner would be expected to occur earlier in the trafficking pathway, perhaps at the level of the trans-Golgi network. Nonetheless, the class of IFT proteins as well as pericentrin are excellent candidate partners for directing PC-2 to cilia. Several other binding partners for PC-2 have been described including actin cytoskeleton interacting protein CD2AP (Lehtonen et al., 2000), PIGEA-14 which modulates PC-2 distribution within the Golgi (Hidaka et al., 2004) and PACS-1 and PACS-2 which are involved in trafficking of PC-2 between ER and Golgi and plasma membrane compartments (Kottgen et al., 2005). These latter three partners bind in the COOH-terminus of PC-2 and are unlikely to play a direct role in the mechanism of trafficking by the RVXP motif.

PC-2 Trafficking to Cilia is Independent of PC-1

The most common and severe form of ADPKD results from mutations in PKD1. Genetic studies suggest that PKD1 and PKD2 function in the same genetic pathway and biochemical studies support the hypothesis that PC-1 and PC-2 work together in regulating key functions in polarized, lumen forming epithelial tissues (Barr et al., 2001; Hanaoka et al., 2000; Wu et al., 2002). The cation channel activity of PC-2 is critical to the function of the polycystin signaling complex as evidenced by the pathogenic amino acid substitution mutation, D511V, that results in loss of channel activity (Reynolds et al., 1999; Koulen et al., 2002) without affecting interaction with PC-1 or to cilia localization (unpublished observations). Applicant's bilayer experimental data supports the view that PC-2, like other TRP channels, can form a functional channel complex without PC-1 (Koulen et al., 2002; Gonzalez-Perrett et al., 2001). In cilia, PC-1 is thought exert control of the PC-2 channel that ultimately renders PC-2 ineffective in ADPKD patients who have PKD1 mutations resulting in loss of PC-1 (Nauli et al., 2003). One proposed level at which PC-1 exerts this control has been hypothesized to be in the proper trafficking of PC-2 (Hanaoka et al., 2000; Nauli et al., 2003).

Previous studies had suggested that co-assembly with PC-1 is required for surface expression of PC-2 (Hanaoka et al., 2000) and that PC-2 is not expressed in cilia in the absence of PC-1 (Nauli et al., 2003). Applicant's current studies indicate that PC-1 is not required for trafficking of PC-2 to cilia. Full length, heterologously expressed PC-2 can leave the ER and reach the cilia without PC-1. Furthermore, PC-2 is expressed in cilia in kidney cysts that result from null mutations in PKD1. Given the evidence of functional interdependence for the two polycystins, loss of PC-1 likely results in loss of critical regulatory signals rendering PC-2 ineffective despite expression of the latter in cilia. It is also possible that PC-1 participates more actively in the channel function, perhaps forming part of a heteromeric channel with PC-2. However, data cited above do support the hypothesis that PC-2 has channel function independently of PC-1. While it is nominally possible that the role of PC-1 is to turn off a constitutively active PC-2 channel, this seems very unlikely given that the phenotype from loss of PC-1 is indistinguishable from that from loss of PC-2. Therefore, the most likely formulation is that activation of the PC-1 receptor protein by relevant stimuli results in activation of the PC-2 cation channel which in turn propagates a signal that uses Ca²⁺ as a second messenger. It follows that selective PC-2 agonists may be able to reconstitute polycystin signaling in patients with PKD1 mutations by bypassing the receptor defect and acting directly on the effector channel protein. Work described herein supports the role of selective PC-2 channel agonist as effective therapeutic agents for the most common and severe forms of ADPKD.

### REFERENCES FOR EXAMPLES 1-6

1. Barr, M. M., J. DeModena, D. Braun, C. Q. Nguyen, D. H. Hall, and P. W. Sternberg. 2001. The Caenorhabditis elegans autosomal dominant polycystic kidney disease gene homologs lov-1 and pkd-2 act in the same pathway. Curr. Biol. 11: 1341-1346.
2. Bouck, G. B. 1971. The structure, origin, isolation, and composition of the tubular mastigonemes of the Ochromas flagellum. J Cell Biol. 50:362-384.
3. Cai, Y., G. Anyatonwu, D. Okuhara, K. B. Lee, Z. Yu, T. Onoe, C. L. Mei, Q. Qian, L. Geng, R. Wiztgall, B. E. Ehrlich, and S. Somlo. 2004. Calcium dependence of polycystin-2 channel activity is modulated by phosphorylation at Ser812. J. Biol. Chem. 279:19987-19995.
4. Cai, Y., Y. Maeda, A. Cedzich, V. E. Torres, G. Wu, T. Hayashi, T. Mochizuki, J. H. Park, R. Witzgall, and S. Somlo. 1999. Identification and characterization of polycystin-2, the PKD2 gene product. J. Biol. Chem. 274:28557-28565.
5. Chauvet, V., X. Tian, H. Husson, D. H. Grimm, T. Wang, T. Hieseberger, P. Igarashi, A. M. Bennett, O. Ibraghimov-Beskrovnaya, S. Somlo, and M. J. Caplan. 2004. Mechanical stimuli induce cleavage and nuclear translocation of the polycystin-1 C terminus. J Clin. Invest 114:1433-1443.
6. Chen, X. Z., P. M. Vassilev, N. Basora, J. B. Peng, H. Nomura, Y. Segal, E. M. Brown, S. T. Reeders, M. Hediger, and J. Zhou. 1999. Polycystin-L is a calcium-regulated cation channel permeable to calcium ions. Nature 401:383-386.
7. Collawn, J. F., M. Stangel, L. A. Kuhn, V. Esekogwu, S. Q. Jing, I. S. Trowbridge, and J. A. Tainer. 1990. Transferrin receptor internalization sequence YXRF implicates a tight turn as the structural recognition motif for endocytosis. Cell 63:1061-1072.
8. Davila, S., L. Furu, A. G. Gharavi, X. Tian, T. Onoe, Q. Qian, A. Li, Y. Cai, P. S. Kamath, B. F. King, P. J. Azurmendi, P. Tahvanainen, H. Kaariainen, K. Hockerstedt, O. Devuyst, Y. Pirson, R. S. Martin, R. P. Lifton, E. Tahvanainen, V. E. Torres, and S. Somlo. 2004. Mutations in SEC63 cause autosomal dominant polycystic liver disease. Nat. Genet. 36:575-577.
9. Deane, J. A., D. G. Cole, E. S. Seeley, D. R. Diener, and J. L. Rosenbaum. 2001. Localization of intraflagellar transport protein IFT52 identifies basal body transitional fibers as the docking site for IFT particles. Curr. Biol. 11:1586-1590.
10. Drenth, J. P., R. H. Te Morsche, R. Smink, J. S. Bonifacino, and J. B. Jansen. 2003. Germline mutations in PRKCSH are associated with autosomal dominant polycystic liver disease. Nat. Genet.
11. Ersfeld, K. and K. Gull. 2001. Targeting of cytoskeletal proteins to the flagellum of Trypanosoma brucei. J. Cell Sci. 114:141-148.
12. Godsel, L. M. and D. M. Engman. 1999. Flagellar protein localization mediated by a calcium-myristoyl/palmitoyl switch mechanism. EMBO J. 18:2057-2065.
13. Gonzalez-Perrett, S., K. Kim, C. Ibarra, A. E. Damiano, E. Zotta, M. Batelli, P. C. Harris, I. L. Reisin, M. A. Arnaout, and H. F. Cantiello. 2001. From the Cover: Polycystin-2, the protein mutated in autosomal dominant polycystic kidney disease (ADPKD), is a Ca2+-permeable nonselective cation channel. PNAS 98:1182-1187.
14. Gottardi, C. J., L. A. Dunbar, and M. J. Caplan. 1995. Biotinylation and assessment of membrane polarity: caveats and methodological concerns. American Journal of Physiology 268:F285-F295.
15. Hanaoka, K., F. Qian, A. Boletta, A. K. Bhunia, K. Piontek, L. Tsiokas, V. P. Sukhatme, W. B. Guggino, and G. G. Germino. 2000. Co-assembly of polycystin-1 and -2 produces unique cation-permeable currents. Nature 408:990-994.
16. Hateboer, N., M. A. Dijk, N. Bogdanova, E. Coto, A. K. Saggar-Malik, J. L. San Millan, R. Torra, M. Breuning, and D. Ravine. 1999. Comparison of phenotypes of polycystic kidney disease types 1 and 2. European PKD1-PKD2 Study Group. Lancet 353:103-107.
17. Hidaka, S., V. Konecke, L. Osten, and R. Witzgall. 2004. PIGEA-14, a novel coiled-coil protein affecting the intracellular distribution of polycystin-2. J Biol. Chem. 279:35009-35016.
18. Jing, S. Q., T. Spencer, K. Miller, C. Hopkins, and I. S. Trowbridge. 1990. Role of the human transferrin receptor cytoplasmic domain in endocytosis: localization of a specific signal sequence for internalization. J. Cell Biol. 110:283-294.
19. Jurczyk, A., A. Gromley, S. Redick, A. J. San, G. Witman, G. J. PazOur, D. J. Peters, and S. Doxsey. 2004. Pericentrin forms a complex with intraflagellar transport proteins and polycystin-2 and is required for primary cilia assembly. J Cell Biol. 166:637-643.
20. Kim, K., I. Drummond, O. Ibraghimov-Beskrovnaya, K. Klinger, and M. A. Arnaout. 2000. Polycystin 1 is required for the structural integrity of blood vessels. Proc. Natl. Acad. Sci. U.S. A 97:1731-1736.
21. Kottgen, M., T. Benzing, T. Simmen, R. Tauber, B. Buchholz, S. Feliciangeli, T. B. Huber, B. Schermer, A. Kramer-Zucker, K. Hopker, K. C. Simmen, C. C. Tschucke, R. Sandford, E. Kim, G. Thomas, and G. Walz. 2005. Trafficking of TRPP2 by PACS proteins represents a novel mechanism of ion channel regulation. EMBO J. 24:705-716.
   22. Koulen, P., Y. Cai, L. Geng, Y. Maeda, S. Nishimura, R. Witzgall, B. E. Ehrlich, and S. Somlo. 2002. Polycystin-2 is an intracellular calcium release channel. Nat. Cell Biol. 4:191-197.
23. Kozminski, K. G., K. A. Johnson, P. Forscher, and J. L. Rosenbaum. 1993. A motility in the eukaryotic flagellum unrelated to flagellar beating. Proc. Natl. Acad. Sci. U.S. A 90:5519-5523.
24. Lehtonen, S., A. Ora, V. M. Olkkonen, L. Geng, M. Zerial, S. Somlo, and E. Lehtonen. 2000. In vivo interaction of the adapter protein CD2-associated protein with the type 2 polycystic kidney disease protein, polycystin-2. J. Biol. Chem. 275:32888-32893.
25. Li, A., S. Davila, L. Furu, Q. Qian, X. Tian, P. S. Kamath, B. F. King, V. E. Torres, and S. Somlo. 2003. Mutations in PRKCSH Cause Isolated Autosomal Dominant Polycystic Liver Disease. Am J Hum Genet. 72:691-703.
26. Li, Q., N. Montalbetti, P. Y. Shen, X. Q. Dai, C. I. Cheeseman, E. Karpinski, G. Wu, H. F. Cantiello, and X. Z. Chen. 2005. Alpha-actinin associates with polycystin-2 and regulates its channel activity. Hum. Mol. Genet. 14:1587-1603.
27. Lin, F., T. Hiesberger, K. Cordes, A. M. Sinclair, L. S. Goldstein, S. Somlo, and P. Igarashi. 2003. Kidney-specific inactivation of the KIF3A subunit of kinesin-II inhibits renal ciliogenesis and produces polycystic kidney disease. Proc. Natl. Acad. Sci. U.S. A 100:5286-5291.
28. Lu, W., B. Peissel, H. Babakhanlou, A. Pavlova, L. Geng, X. Fan, C. Larson, G. Brent, and J. Zhou. 1997. Perinatal lethality with kidney and pancreas defects in mice with a targeted Pkd1 mutation. Nat. Genet. 17:179-181.
29. Lu, W., X. Shen, A. Pavlova, M. Lakkis, C. J. Ward, L. Pritchard, P. C. Harris, D. R. Genest, A. R. Perez-Atayde, and J. Zhou. 2001. Comparison of Pkd1-targeted mutants reveals that loss of polycystin-1 causes cystogenesis and bone defects. Hum. Mol. Genet. 10:2385-2396.
30. Luo, Y., P. M. Vassilev, X. Li, Y. Kawanabe, and J. Zhou. 2003. Native polycystin 2 functions as a plasma membrane Ca2+-permeable cation channel in renal epithelia. Mol. Cell Biol. 23:2600-2607.
31. McGrath, J., S. Somlo, S. Makova, X. Tian, and M. Brueckner. 2003. Two populations of node monocilia initiate left-right asymmetry in the mouse. Cell 114:61-73.
32. Meder, D., A. Shevchenko, K. Simons, and J. Fullekrug. 2005. Gp135/podocalyxin and NHERF-2 participate in the formation of a preapical domain during polarization of MDCK cells. J Cell Biol. 168:303-313.
33. Mochizuki, T., G. Wu, T. Hayashi, S. L. Xenophontos, B. Veldhuisen, J. J. Saris, D. M. Reynolds, Y. Cai, P. A. Gabow, A. Pierides, W. J. Kimberling, M. H. Breuning, C. C. Delta S, D. J. Peters, and S. Somlo. 1996. PKD2, a gene for polycystic kidney disease that encodes an integral membrane protein. Science 272:1339-1342.
34. Nakamura, N., C. Rabouille, R. Watson, T. Nilsson, N. Hui, P. Slusarewicz, T. E. Kreis, and G. Warren. 1995. Characterization of a cis-Golgi matrix protein, GM130. J Cell Biol. 131:1715-1726.
35. Nasser, M. I. and S. M. Landfear. 2004. Sequences required for the flagellar targeting of an integral membrane protein. Mol. Biochem. Parasitol. 135:89-100.
36. Nauli, S. M., F. J. Alenghat, Y. Luo, E. Williams, P. Vassilev, X. Li, A. E. Elia, W. Lu, E. M. Brown, S. J. Quinn, D. E. Ingber, and J. Zhou. 2003. Polycystins 1 and 2 mediate mechanosensation in the primary cilium of kidney cells. Nat. Genet.
37. Nir, I., D. Cohen, and D. S. Papermaster. 1984. Immunocytochemical localization of opsin in the cell membrane of developing rat retinal photoreceptors. J Cell Biol. 98:1788-1795.
38. Nomura, H., A. E. Turco, Y. Pei, L. Kalaydjieva, T. Schiavello, S. Weremowicz, W. Ji, C. C. Morton, M. Meisler, S. T. Reeders, and J. Zhou. 1998. Identification of PKDL, a novel polycystic kidney disease 2-like gene whose murine homologue is deleted in mice with kidney and retinal defects. J. Biol. Chem. 273:25967-25973.
39. O'Toole, E. T., T. H. Giddings, J. R. McIntosh, and S. K. Dutcher. 2003. Three-dimensional organization of basal bodies from wild-type and delta-tubulin deletion strains of Chlamydomonas reinhardtii. Mol. Biol. Cell 14:2999-3012.
40. Pan, J., Q. Wang, and W. J. Snell. 2005. Cilium-generated signaling and cilia-related disorders. Lab Invest 85:452-463.
   41. PazOur, G. J. 2004. Intraflagellar transport and cilia-dependent renal disease: the ciliary hypothesis of polycystic kidney disease. J Am Soc Nephrol 15:2528-2536.
42. PazOur, G. J., B. L. Dickert, Y. Vucica, E. S. Seeley, J. L. Rosenbaum, G. B. Witman, and D. G. Cole. 2000. Chlamydomonas IFT88 and its mouse homologue, polycystic kidney disease gene tg737, are required for assembly of cilia and flagella. J Cell Biol. 151:709-718.
43. PazOur, G. J., J. T. San Agustin, J. A. Follit, J. L. Rosenbaum, and G. B. Witman. 2002. Polycystin-2 localizes to kidney cilia and the ciliary level is elevated in orpk mice with polycystic kidney disease. Curr. Biol. 12:R378-R380.
44. Pennekamp, P., C. Karcher, A. Fischer, A. Schweickert, B. Skryabin, J. Horst, M. Blum, and B. Dworniczak. 2002. The ion channel polycystin-2 is required for left-right axis determination in mice. Curr. Biol. 12:938-943.
45. Praetorius, H. A. and K. R. Spring. 2001. Bending the MDCK cell primary cilium increases intracellular calcium. J Membr. Biol. 184:71-79.
46. Qian, F., A. Boletta, A. K. Bhunia, H. Xu, L. Liu, A. K. Ahrabi, T. J. Watnick, F. Zhou, and G. G. Germino. 2002. Cleavage of polycystin-1 requires the receptor for egg jelly domain and is disrupted by human autosomal-dominant polycystic kidney disease 1-associated mutations. Proc. Natl. Acad. Sci. U.S. A 99:16981-16986.
47. Qian, F., F. J. Germino, Y. Cai, X. Zhang, S. Somlo, and G. G. Germino. 1997. PKD1 interacts with PKD2 through a probable coiled-coil domain. Nat. Genet. 16:179-183.
48. Qian, F., T. J. Watnick, L. F. Onuchic, and G. G. Germino. 1996. The molecular basis of focal cyst formation in human autosomal dominant polycystic kidney disease type I. Cell 87:979-987.
49. Reynolds, D. M., T. Hayashi, Y. Cai, B. Veldhuisen, T. J. Watnick, X. M. Lens, T. Mochizuki, F. Qian, Y. Maeda, Z. Li, R. Fossdal, E. Coto, G. Q. Wu, M. H. Breuning, G. G. Germino, D. J. M. Peters, and S. Somlo. 1999. Aberrant splicing in the PKD2 gene as a cause of polycysitic kidney disease. J. Am. Soc. Nephrol. 10:2342-2351.
50. Rosenbaum, J. L. and G. B. Witman. 2002. Intraflagellar transport. Nat. Rev. Mol. Cell Biol. 3:813-825.
51. Rundle, D. R., G. Gorbsky, and L. Tsiokas. 2004. PKD2 interacts and co-localizes with mDial to mitotic spindles of dividing cells: role of mDial IN PKD2 localization to mitotic spindles. J. Biol. Chem. 279:29728-29739.
52. Satir, B., W. S. Sale, and P. Satir. 1976. Membrane renewal after dibucaine deciliation of Tetrahymena. Freeze-fracture technique, cilia, membrane structure. Exp. Cell Res. 97:83-91.
53. Scheffers, M. S., B. P. van der, W. A. van de, E. J. van, M. H. Breuning, H. E. de, and D. J. Peters. 2004. Altered distribution and co-localization of polycystin-2 with polycystin-1 in MDCK cells after wounding stress. Exp. Cell Res. 292:219-230.
54. Sun, Z., A. Amsterdam, G. J. PazOur, D. G. Cole, M. S. Miller, and N. Hopkins. 2004. A genetic screen in zebrafish identifies cilia genes as a principal cause of cystic kidney. Development 131:4085-4093.
55. Thomson, R. B., S. Mentone, R. Kim, K. Earle, E. Delpire, S. Somlo, and P. S. Aronson. 2003. Histopathological analysis of renal cystic epithelia in the Pkd2WS25/− mouse model of ADPKD. Am J Physiol Renal Physiol 285:F870-F880.
56. Tsiokas, L., E. Kim, T. Arnould, V. P. Sukhatme, and G. Walz. 1997. Homo- and heterodimeric interactions between the gene products of PKD1 and PKD2. Proc. Natl. Acad. Sci. U.S.A. 94:6965-6970.
57. Tull, D., J. E. Vince, J. M. Callaghan, T. Naderer, T. Spurck, G. I. McFadden, G. Currie, K. Ferguson, A. Bacic, and M. J. McConville. 2004. SMP-1, a member of a new family of small myristoylated proteins in kinetoplastid parasites, is targeted to the flagellum membrane in Leishmania. Mol. Biol. Cell 15:4775-4786.
58. Witzgall, R. 2005. Polycystin-2—an intracellular or plasma membrane channel Naunyn Schmiedebergs Arch. Pharmacol. 371:342-347.
59. Wu, G., V. D'Agati, Y. Cai, G. Markowitz, J. H. Park, D. M. Reynolds, Y. Maeda, T. C. Le, H. J. Hou, R. Kucherlapati, W. Edelmann, and S. Somlo. 1998a. Somatic inactivation of Pkd2 results in polycystic kidney disease. Cell 93:177-188.
60. Wu, G., G. S. Markowitz, L. Li, V. D. D'Agati, S. M. Factor, L. Geng, S. Tibara, J. Tuchman, Y. Cai, J. H. Park, J. van Adelsberg, H. Hou, Jr., R. Kucherlapati, W. Edelmann, and S. Somlo. 2000. Cardiac defects and renal failure in mice with targeted mutations in Pkd2. Nat. Genet. 24:75-78.
61. Wu, G., X. Tian, S. Nishimura, G. S. Markowitz, V. D'Agati, P. J. Hoon, L. Yao, L. Li, L. Geng, H. Zhao, W. Edelmann, and S. Somlo. 2002. Trans-heterozygous Pkd1 and Pkd2 mutations modify expression of polycystic kidney disease. Hum Mol Genet 11:1845-1854.
62. Wu, G. Q., T. Hayashi, J. H. Park, M. P. Dixit, D. M. Reynolds, L. Li, Y. Cai, M. Coca-Prados, and S. Somlo. 1998b. Identification of the human PKD2-related cDNA, PKD2L: tissue-specific expression and linkage mapping on chromosome 10q25. Genomics 54:564-568.
63. Yoder, B. K., X. Hou, and L. M. Guay-Woodford. 2002a. The polycystic kidney disease proteins, polycystin-1, polycystin-2, polaris, and cystin, are co-localized in renal cilia. J. Am. Soc. Nephrol. 13:2508-2516.
64. Yoder, B. K., A. Tousson, L. Millican, J. H. Wu, C. E. Bugg, Jr., J. A. Schafer, and D. F. Balkovetz. 2002b. Polaris, a protein disrupted in orpk mutant mice, is required for assembly of renal cilium. Am J Physiol Renal Physiol 282:F541-F552.
65. Zhang, Q., P. D. Taulman, and B. K. Yoder. 2004. Cystic kidney diseases: all roads lead to the cilium. Physiology. (Bethesda.) 19:225-230.
   Materials and Methods

The following materials and methods were used in Examples 7 and 8.

A) Cells and Reagents

The Pkd1^{-/-} (PN24), Pkd2^{+/-} (3B3) and Pkd2^{-/-} (2D2) murine cell lines were derived from knockout and transgenic mice as previously reported (Wu, et al., 1998, Cell, 93:177-88; Wu, et al., 2000, Nat. Genet, 24:75-8; Wu, et al., 2002, Hum Mol Genet, 11:1845-54 as well as unpublished mice produced by Applicant). The PKD2-Rex cell line was made by stable integration of untagged PKD2 under hygromycin selection into the 2D2 Pkd2^{-/-} cell line background. Antibodies used included anti-polycystin-2 (YCC2, Cai, et al. 1999, J Biol Chem, 274:28557-65), cleaved (active) caspase-3 (Cell Signaling Technology) and p21 (BD Biosciences). Triptolide was obtained from Sinobest Inc. (China) and purity was 99% as determined by HPLC. DMSO was used to dissolve triptolide and was then directly added into culture media for all experiments. Triptolide was tritiated by Sib Tech, Inc. (Newington, Conn.) and resuspended in ethanol to a specific activity of 4-6 Ci/mmol. Purity was >95% as confirmed by RP-HPLC on a Hypersil C18 column and by TLC on both C18 and silica gel.

B) Calcium Imaging

Cells were plated on coverslips and loaded with Fluo-4 (Molecular Probes) diluted in DMSO/pluronic for 30 minutes prior to imaging. Cells were perfused with a calcium imaging buffer (HEPES, NaCl, KCl, MgSO₄ and CaCl₂) ±100 nM triptolide. All cell traces are indicative of individual cellular fluorescence and calcium release. Data is presented as change of fluorescence over baseline control (before triptolide addition).

C) Immunoblotting and Immunofluorescence

Total cell lysates (0.5% Triton X-100, 50 mM Tris pH 7.4, 150 mM NaCl, 500 mM EDTA) were prepared for Western blot analysis and samples were run out by SDS-PAGE as per manufacturers' protocols. Brightfield images of cells were taken using a 10× or 40× objective. Confocal microscopy (40×) was used for immunofluorescence imaging of polycystin-2.

D) In Vivo Murine Experiments

As per approved IACUC animal protocol, Pkd1^{+/-}/Pkd1^{+/-} mice were mated and pregnant mice were divided into control (DMSO) or experimental (triptolide) groups. A total volume of 100 μl of PBS with no more than 5% DMSO or DMSO/0.07 mg/kg/day triptolide was injected i.p. with a 28 G ½ insulin syringe. Mice were weighed and injected starting at E10.5 until birth. All pups were examined for length and developmental staging such as whisker formation. Kidneys were harvested, weighed, and fixed in 4% paraformaldehyde before histological preparation.

E) Histological Examination

Kidneys were prepared by sagittal cross-sectioning and hematoxylin and eosin staining. All kidneys were photographed under the same magnification (4×) and cystic burden was computed using Image J analysis software (NIH). The area of cysts within the total area of the kidney (pixels) was calculated as a final percentage of cystic burden in the kidney. Immunohistochemical analysis of active caspase-3 was completed as per manufacturer's protocol.

F) Triptolide Binding Protein Purification

Five liters of HeLa—S cells (National Cell Culture Center) were labeled with a mixture of [³H]-triptolide as well as unlabelled triptolide for one hour at 37° C. Cells were harvested and washed 5× in cold PBS. The cell pellet was resuspended in hypolysis buffer (10 mM HEPES pH7.9, 10 mM KCl, 0.1 mM EDTA, Complete™ protease inhibitors (Roche), sodium orthovanadate, and DTT) and sheared through a syringe and needle. The supernatant was discarded and the pellet was resolubilized in lysis buffer containing 1% Triton X-100. The membrane fraction was subjected to further purification beginning with binding to the anion exchange resin DE-52 (Whatman). Final elution was completed with 0.3 M NaCl, and then passed through a size exclusion column with 100 kD cutoff (Amicon). The retentate was collected and bound to a Con A Sepharose (GE Healthcare) resin. The flow-through was collected and concentrated by passing over a 100 kD size exclusion column where the retentate was again collected and bound to Heparin Sepharose resin (GE Healthcare). Triptolide binding proteins were eluted with the addition of 1 M ammonium sulfate and 0.1% Triton X-100 and immediately bound to the hydrophobic resin Butyl Sepharose (GE Healthcare). Elution was performed using a no salt buffer (10 mM HEPES pH 7.4, 0.1 mM EDTA) with 1% triton X-100 and 2 mM EGTA. The eluant was subjected to a final concentration over a 100 kD size exclusion column followed by FPLC over a MonoQ anion exchange column. A step gradient of 0.0-1.0 M NaCl (10 mM HEPES pH 7.4, 0.1 mM EDTA, 0.5 M DTT) was run over the MonoQ column. 500 μl fractions were collected and the majority of [³H]-triptolide binding activity was observed between 0.3 M and 0.4 M NaCl. The corresponding fractions were concentrated and run out on by 8% SDS-PAGE and stained with Coomassie Blue. Bands were cut out from the gel and prepared for MALDI-TOF analysis. Proteins of interest were identified using Profound peptide mapping (Rockefeller University).

### Example 7

### Studies on Calcium Dependence Reveal Multiple Modes of Action for Triptolide

Triptolide, a diterpene triepoxide isolated from the traditional Chinese medicinal vine, Trypterygium wilfordii hook f., has been shown to induce rapid apoptosis in myriad cancer cell lines and inhibit NFκB transactivation. To understand further the general cellular mechanisms for this therapeutically relevant natural product, binding and biological activities were assessed. Studies showed that triptolide binding was saturable, reversible and primarily localized to cell membranes. Depletion of calcium enhanced overall binding while differentially modulating biological function. Furthermore, triptolide's structural moieties demonstrated variability in the regulation of cell death versus inhibition of NFκB transactivation. These results implicate triptolide in the manipulation of at least two distinct cellular pathways with differing requirements for calcium and effective triptolide concentration in order to elicit each particular biological function.

The present invention is based, at least in part, on the discovery that agonists of PC-2, such as triptolide and/or triptolide derivatives (e.g., analogs), are effective in slowing or inhibiting growth of kidney cyst cells and in regulating calcium signaling. As described herein, a large-scale protein purification strategy was designed to facilitate the identity of putative triptolide binding protein(s). Following chromatographic protein fractionation, SDS-PAGE separation, and MALDI-MS analysis, a 110 kD band was identified as polycystin-2 and served as a potential biological target for triptolide activity. Applicant demonstrated a calcium dependent effect on triptolide binding and function and showed that triptolide can arrest cell growth or induce apoptosis, depending on the concentration at which it is administered. Based on triptolide's ability to modulate cell growth or death, as based upon its anti-tumor effects, and a putative mechanistic function through polycystin-2 channel activity, Applicant assessed the therapeutic efficacy of triptolide in a model of ADPKD. Triptolide and triptolide derivatives are used as examples of PC-2 agonists which can regulate calcium influx, arrest or slow cell growth, or reduce or slow cyst progression. One of ordinary skill in the art will readily recognize that other PC-2 agonists can be identified using the methods as described herein.

1. [³H]-Triptolide Binding is Reversible and Associates with Cell Membranes.

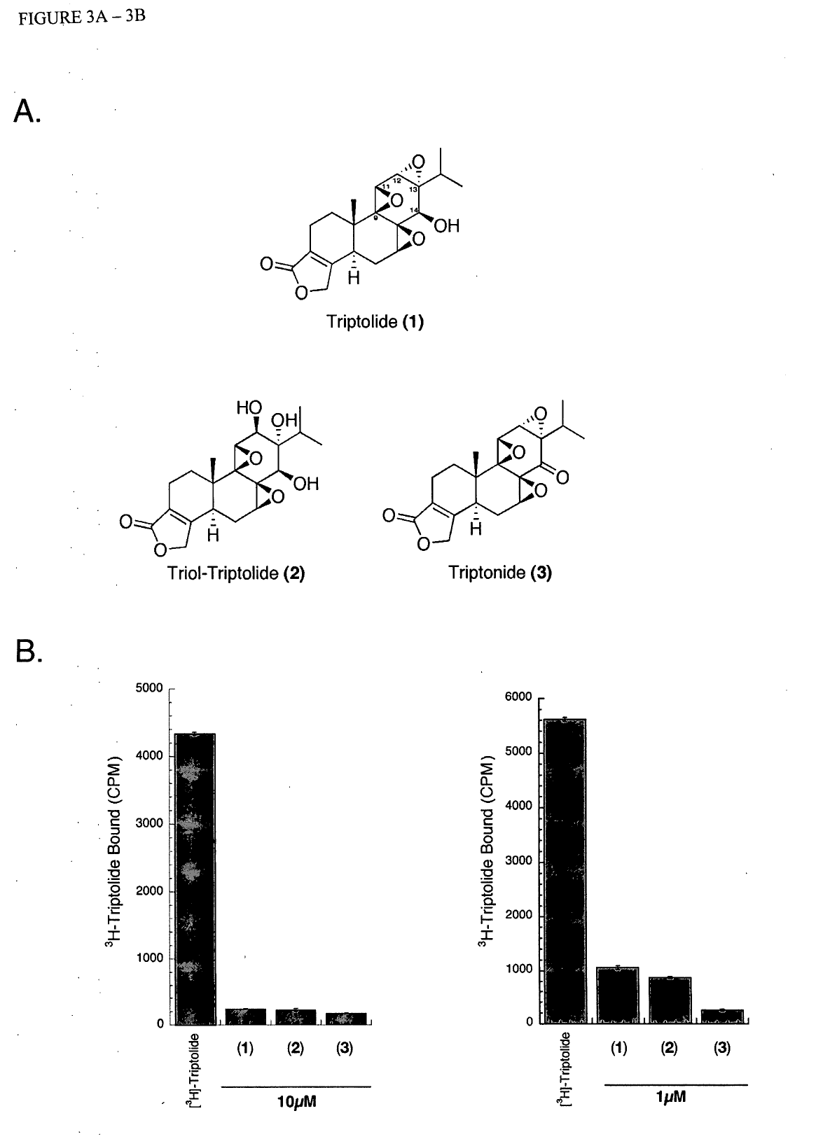

To gain insight into triptolide's mechanism of action Applicant sought to determine its specific binding activity utilizing a system employing [³H]-triptolide. In addition to competition with 1 μM or 10 μM unlabeled triptolide, Applicant also measured the binding affinities of triptolide analogs (FIG. 3A). These analogs have been previously described and differ structurally from triptolide in the hydrolysis of the 12,13 epoxide (2), and formation of the ketone at the C-14 hydroxyl (3) (FIG. 3A). HeLa cells readily bound [³H]-triptolide during an hour-long incubation and binding was significantly competed with pre-treatment of an excess of unlabeled triptolide (1-10 μM) (FIG. 3B). At either 1 or 10 μM, both triptolide analogs showed near total displacement of [³H]-triptolide (FIG. 3B). The effective interaction of these analogs with a presumed triptolide binding entity led us to their use in subsequent experiments addressing the mode of triptolide's biological functions.

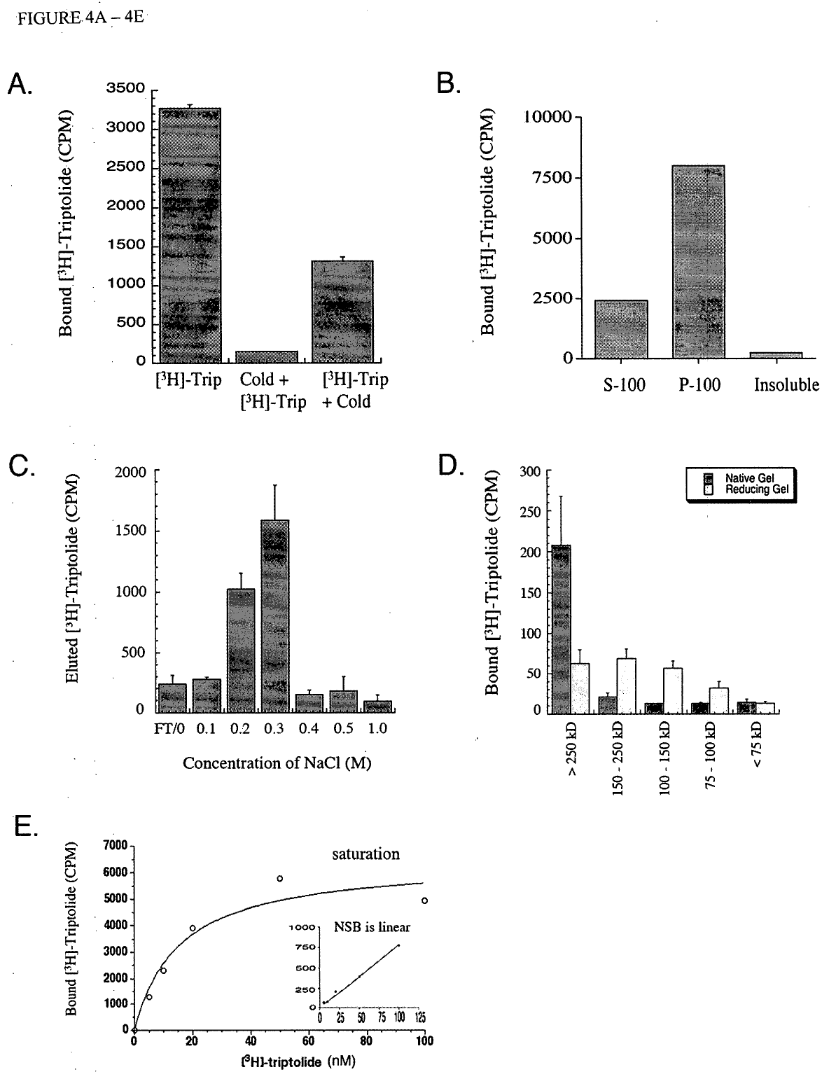

To address first the nature of triptolide binding within the cell, Applicant determined this interaction was reversible as [³H]-triptolide labeling was out competed by subsequent addition of excess unlabeled triptolide (FIG. 4A). An additional labeling experiment followed by cellular fractionation indicated that [³H]-triptolide binds predominantly to the membrane fraction (P-100) of the cell (FIG. 4B). Although 25% of the total cellular counts (i.e., representing bound triptolide) were found in the cytosolic fraction, it is uncertain whether this is specific binding or simply that free triptolide had dissociated from its binding protein due to experimental manipulation during fractionation. Since the data thus far had only shown binding within whole cells, Applicant next determined if this triptolide binding protein could be further characterized and/or enriched by its association with chromatographic reagents. HeLa cells were once again labeled with [³H]-triptolide and total cell lysates were then passed over the anion exchange resin DEAE. Batch elutions with increasing NaCl concentrations were used to disassociate interactions of variable charge. Substantial elution of [³H]-triptolide was not seen in the flow-through or with several salt-free washes and in fact did not begin to elute until the addition of 0.2 M NaCl (FIG. 4C). Additionally, free [³H]-triptolide diluted in lysis buffer and then passed over this resin eluted in the flow-through or during salt-free washes demonstrating that triptolide alone does not interact with this chromatographic media. Importantly, Applicant also observed that [³H]-triptolide would only bind to intact cells in culture but would not bind to any component of a total cellular lysate as assessed by interaction with the DEAE resin. Negative results for a triptolide binding interaction were also observed with DNA-cellulose and the cation exchange resin SPFF (sulphopropyl).

Another line of evidence for direct protein interaction involved a further separation of [³H]-triptolide labeled membrane preparations. Labeling of cells and total membrane purification by high speed centrifugation followed by detergent resolubilization yielded samples which were run out on 6% polyacrylamide gels under native or denaturing conditions (the [³H]-triptolide lysates were not boiled in either experimental condition). Gel slices were extracted based on molecular weight range, crushed, and water extracted followed by liquid scintillation. Native gel separation indicated that [³H]-triptolide was bound to protein(s) above 250 kD, while denaturing conditions showed a separation of [³H]-triptolide binding in a range from 75 to greater than 250 kD (FIG. 4D). These results suggested that triptolide could be binding to a protein complex that then dissociates upon reducing conditions. These data are also supported by size exclusion assays in which total cell lysates labeled with [³H]-triptolide retain the majority of binding interaction above a molecular weight cutoff of 100 kD.

Based upon the data above, Applicant next sought to determine if triptolide binding was saturable and if more than one protein (or binding site) was being targeted. Determination of the [³H]-triptolide binding affinity (K_{D}) and the binding capacity per cell (Bmax) were calculated using a saturation plot. HeLa cells were cultured to 90% confluency and subsequently treated with 0-100 nM of [³H]-triptolide for one hour. Non-specific binding was defined using 2 μM cold triptolide as competitor. Bmax was calculated to be 99±19 fmol triptolide bound/105 cells of triptolide binding sites. Specific binding was found to be saturable with a KD value of 15.5±0.8 nM, while non-specific binding was linear (non-saturable) (FIG. 4E).

2. Triptolide Binding Activity is Influenced by Extracellular Calcium Concentration.

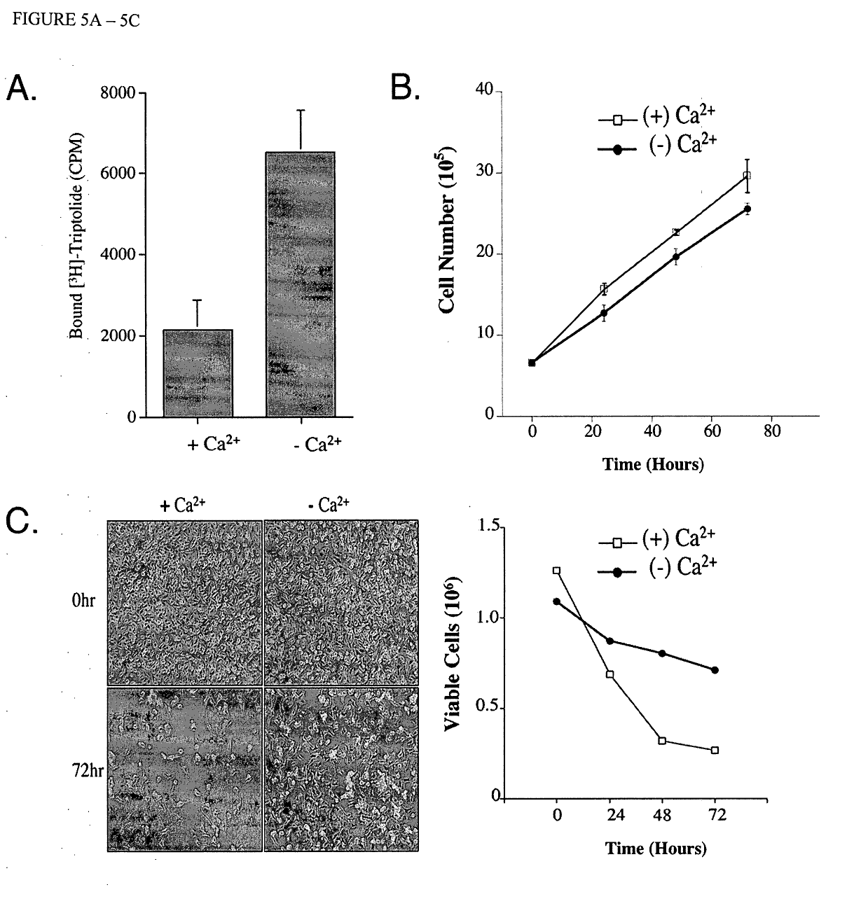

To understand further the nature of triptolide's interaction within the cell, Applicant altered cell culture conditions to examine if [³H]-triptolide binding would be affected. Calcium has been shown to mediate numerous cellular functions, including transcriptional activation of NF-AT and NFκB (Tomida, et al., 2003, EMBO J 22, 3825-3832; Dolmetsch, et al., 1997, Nature 386, 855-858; Dolmetsch, et al., 1998, Nature 392, 933-936). Additionally, it has been established that aberrant calcium signaling can result in cell death (Rizzuto, et al., 2003, Oncogene 22, 8619-8627; Orrenius, et al., 2003, Nat Rev Mol Cell Biol 4, 552-565). Due to triptolide's own link with NFκB and NF-AT and its propensity to induce cell death, Applicant assessed whether triptolide binding could be modulated by free calcium levels. Adherent HeLa cells were cultured in the presence or absence of calcium containing medium for 16 hours before [³H]-triptolide addition. Replicate experiments confirmed that in the absence of extracellular calcium, [³H]-triptolide binding significantly increased between 2-4 fold depending on cell number and density (FIG. 5A). It is also noteworthy that an increase in cell density (not cell number) also increases triptolide binding regardless of calcium levels. Additionally, specific calcium chelation by 10 mM EGTA for one hour in calcium containing medium increased binding by nearly 2 fold. These data indicate that triptolide interaction with its target protein(s) is potentially stabilized or enhanced when calcium levels are low.

3. Triptolide Induced Cell Death is Delayed in the Absence of Calcium.

Since extracellular calcium concentration can influence triptolide binding Applicant sought to determine if the presence of calcium influences the rate of triptolide-mediated apoptosis. To first establish the growth rate of HeLa in media±calcium, cell counts were performed over a 72 hour time course. Although calcium-free media caused cells to detach more easily, the overall growth rates were similar where cell doubling occurred every 24 hours on average (FIG. 5B). For triptolide experiments, cells were initially equilibrated in medium±calcium for 16 hours before the addition of 100 nM triptolide. Cell death was assessed by trypan blue dye exclusion at 24, 48, and 72 hours post drug treatment. In the presence of calcium-containing medium, triptolide induced at least 50% cell death by 24 hours with this trend continuing through later time points (FIG. 5C). In contrast, removal of calcium from the growth medium resulted in a higher proportion of viable cells (FIG. 5C). Following 72 hours of triptolide addition in calcium free media, only 35% of the cells had died indicating that there is a significant delay in this process. These results support a role for calcium in efficient cell death induced by triptolide. There is, however, a likely secondary (albeit slower) mechanism to promote apoptosis as the lack of calcium merely delays but does not eliminate cell death.

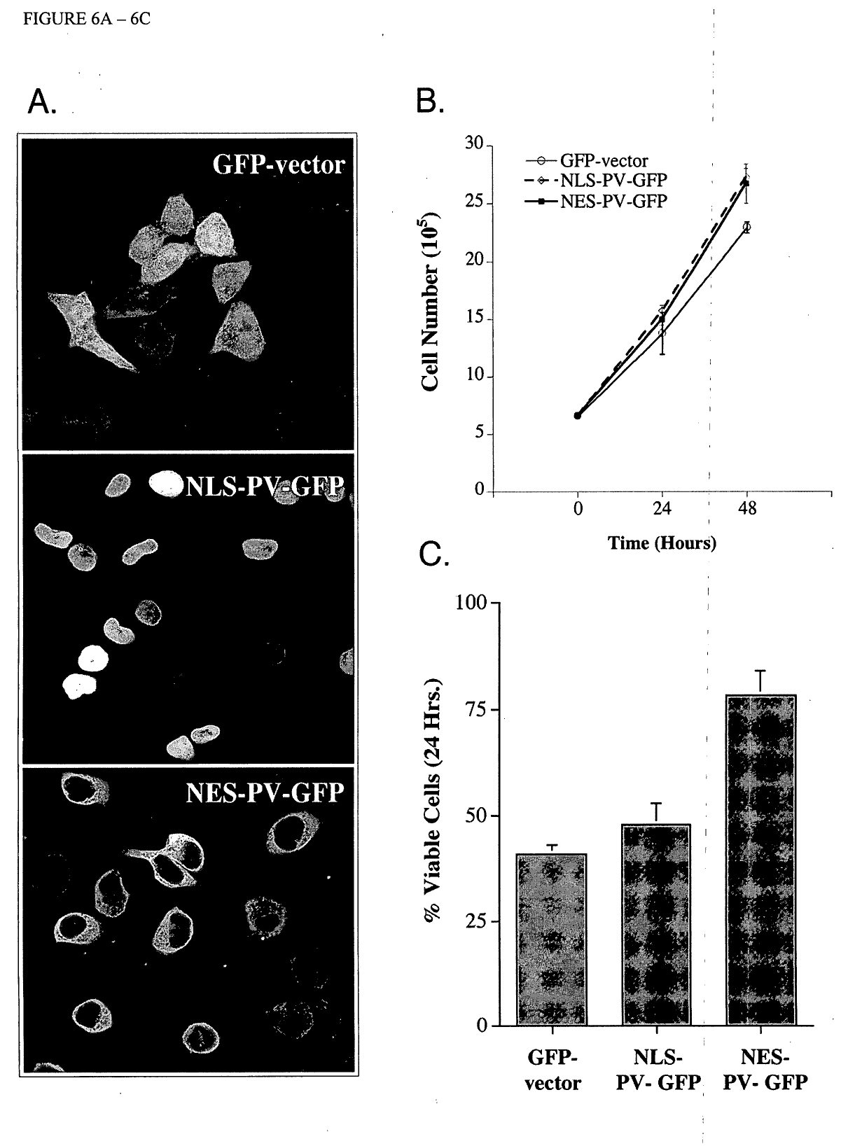

To investigate further the role of calcium in triptolide function Applicant utilized a system to buffer intracellular calcium levels. Various GFP-parvalbumin (PV) fusion proteins can be specifically localized to either the nucleus or the cytoplasm through a nuclear localization or exclusion signal (NLS or NES, respectively) (Pusl, et al., 2002, J Biol Chem 277, 27517-27527). Parvalbumin has two EF-hand calcium binding domains and can efficiently reduce the availability of free calcium in the cell (Pauls, et al., 1996, Biochim Biophys Acta 1306, 39-54). HeLa cells were transiently transfected with the GFP vector control, NES-PV-GFP, or NLS-PV-GFP in calcium containing media and efficient expression of each construct was determined by GFP localization (FIG. 6A). Normal cell growth was first assessed throughout 48 hours with each of the constructs. All transfections resulted in normal growth doubling during the course of the experiment without drug addition (FIG. 6B). For triptolide experiments, cells were transfected and allowed to express the construct for 24 hours before the addition of 100 nM triptolide. After 24 and 48 hours of treatment, cells were assessed for viability. Both control GFP vector and NLS-PV-GFP showed similar apoptosis induction whereby 50% of the cells were rounded and no longer viable after 24 hours (FIG. 6C). In marked contrast, cytosolic parvalbumin (NES-PV-GFP) significantly inhibited triptolide induced cell death at this time point (15-20% apoptosis) (FIG. 6C). This effect was transient however as there was complete cell death in all conditions by 48 hours. Since the parvalbumin buffering experiments are done in the presence of extracellular calcium, the cell can operate normally in that intracellular calcium stores may be refilled. It is reasonable to expect then if calcium homeostasis is being affected (i.e., cytosolic calcium levels increase) by triptolide, then parvalbumin would eventually reach a saturation point. This might explain why the rescue from apoptosis is transient through the 24 hour time point but is lost by 48 hours. These results not only confirm the overall importance of calcium to triptolide function, but more specifically point to cytosolic calcium levels as a mediator in triptolide induced cell death.

4. Inhibition of NFκB Transactivation by Triptolide is Calcium Independent.

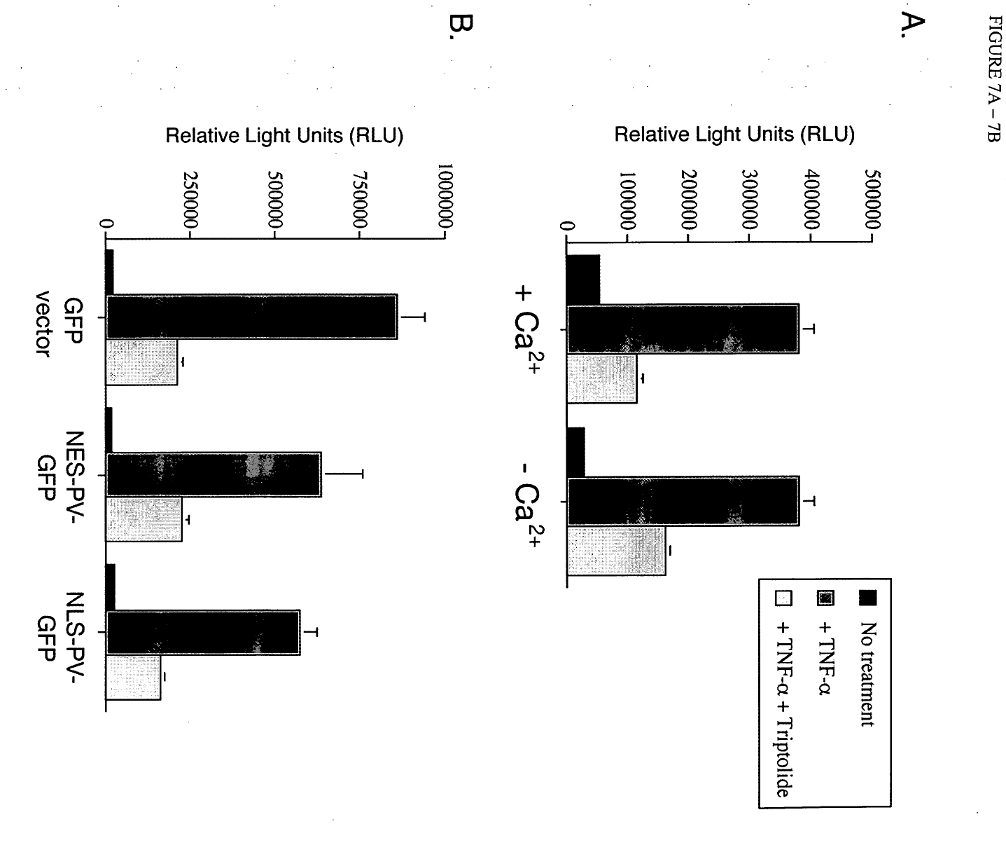

Having established that triptolide induced cell death is dependent upon free calcium concentration, Applicant next determined whether this was also a requirement for the inhibition of NFκB transcription. HeLa cells were transiently transfected with the κB-luciferase reporter construct for 8 hours before being washed and then cultured in the presence or absence of calcium containing medium for 16 hours. Triptolide (100 nM) was pre-incubated with cells for 1 hour before addition of 15 ng/ml of TNF-α for 4 hours. Similar profiles were seen in both the presence and absence of calcium as TNF-α induced NFκB transactivation, while triptolide effectively inhibited it (FIG. 7A).

As an additional experiment, transcriptional activity was also assessed by site-directed calcium buffering. HeLa cells were co-transfected with the κB-luciferase plasmid as well as one of the following constructs: GFP empty vector, NES-PV-GFP, or NLS-PV-GFP and proper GFP localization was confirmed. Cells were grown in the presence of calcium for 24 hours before the addition of 100 nM triptolide and 15 ng/ml TNF-α. NFκB transactivation by TNF-α alone was quite high, although both NES- and NLS-parvalbumin transfected cells showed slightly lower levels of luciferase expression as compared to the vector control. Importantly, triptolide still retained the ability to suppress NFκB transactivation in all experimental conditions (FIG. 7B). These results suggest that while efficient induction of apoptosis by triptolide is calcium dependent, inhibition of NFκB transcriptional control is not.

5. Triptolide Concentration Differentially Effects Cell Death and Inhibition of NFκB.

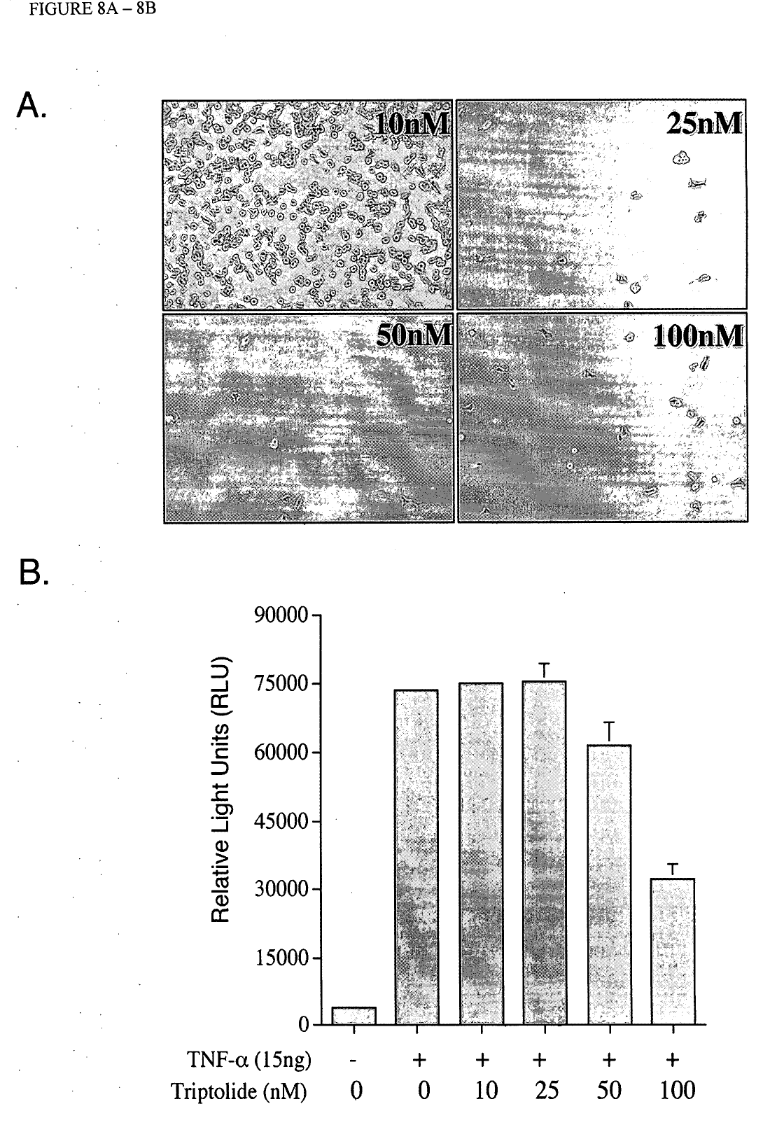

Applicant's results have implicated reversible binding of triptolide to a potential binding protein or complex that can be regulated by calcium. To understand if triptolide function is further separable between cell death and NFκB inhibition, Applicant examined the effect of concentration on these two endpoints. HeLa cells were cultured in the presence of 0, 10, 25, 50, or 100 nM of triptolide and separately assessed for cell death or the ability to suppress NFκB transactivation promoted by TNF-α. Following 24-48 hours of culture, viable cells were recovered and counted. Following 24 hours, triptolide concentrations from 25-100 nM caused greater than 50% of the cells to undergo cell death as assessed by detachment, clumping and the failure to exclude trypan blue dye. After 48 hours, nearly all cells treated within this concentration range of triptolide died (FIG. 8A). While untreated HeLa cells underwent two cycles of division, 10 nM triptolide inhibited cell proliferation but did not induce cell death. This is consistent with previous studies showing that low doses of triptolide cause cell cycle arrest rather than apoptosis (Kiviharju, et al., 2002, Clin Cancer Res 8, 2666-2674). It is also of note that triptolide's action on the cell that ultimately results in cell death is initially reversible, as three to four hours is the minimal incubation time required for commitment to apoptosis.

NFκB transactivation was examined using the κB-luciferase reporter construct. HeLa cells were transiently transfected and pre-treated with 0-100 nM triptolide for one hour prior to TNF-α addition. Cells were assessed for NFκB driven luciferase expression after an additional five hours of incubation, at which time TNF-α had induced transcriptional activity by approximately 15-fold in control cells. Both 10 and 25 nM triptolide did not inhibit TNF-α driven transcriptional activity of NFκB (concentrations shown to inhibit proliferation or induce cell death, respectively), whereas 50 nM suppressed activity by 20%, and 100 nM had the most profound effect with an average of 60% inhibition (FIG. 8B). It is also of note that luciferase activity assayed after 24 hours with 10 nM triptolide+TNF-α still showed greater than a 20-fold induction. Examination of these two biological endpoints at the same chronological time indicates that while 10 nM is efficient at arresting cell growth it cannot inhibit TNF-α induced NFκB transcriptional activity. The results thus far support a divergence of triptolide-mediated functions: the pathway regulating growth arrest/death is more sensitive to triptolide and calcium than the mechanism leading to transcriptional repression of NFκB.

6. Triptolide Analogs Show Differential Abilities to Induce Cell Death or Inhibit NFκB Transactivation.

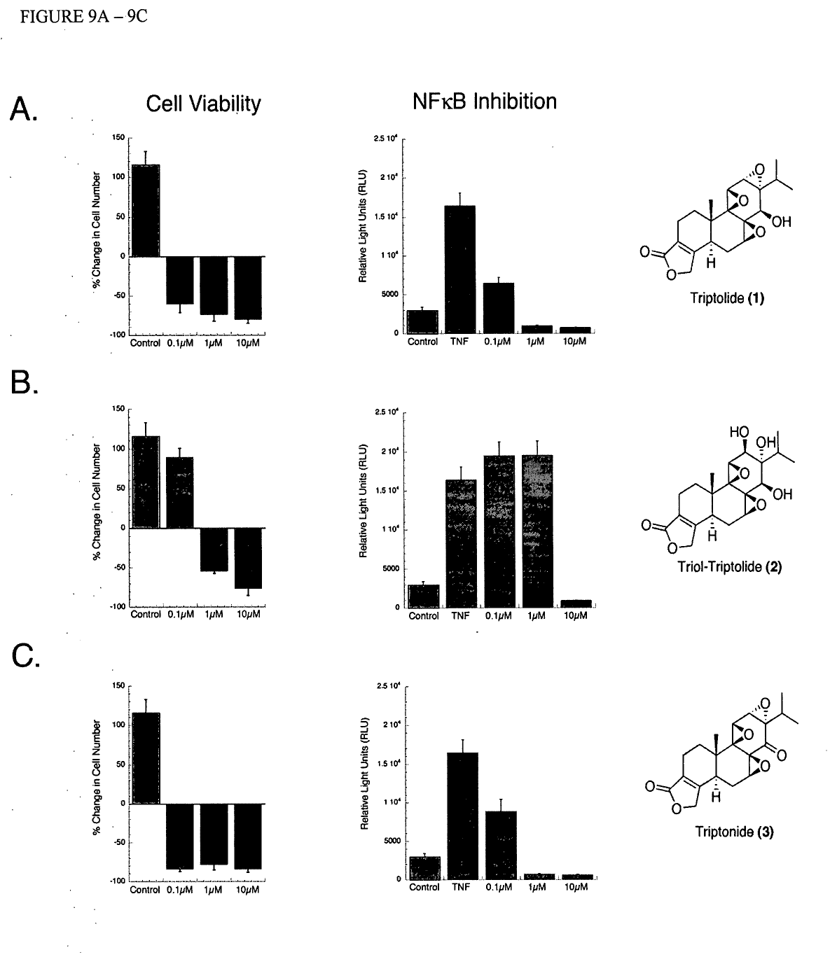

Based upon Applicant's studies examining the concentration dependent effect of triptolide on the two measured biological endpoints, cell death and transcriptional repression, Applicant wanted to determine how modulating triptolide's structure may also discriminate between the two pathways. For cell viability assays, HeLa cells were incubated with 0, 0.1, 1, or 10 μM of each analog or triptolide for 24 hours and then counted using trypan blue dye exclusion. Triptolide, as shown before, effectively induced greater than 50% cell death at 0.1 μM with no significant increase at the higher concentrations (FIG. 9A). NFκB transcriptional inhibition was measured using the κB-luciferase assay as previously described following 5 hours of incubation with each analog (0-10 μM) and TNF-α addition. NFκB inhibition was greater than 60% and attenuated further as triptolide's concentration increased to 1 or 10 μM (FIG. 9A).

Upon disruption of the 12,13 epoxide in analog (2), differential effects were seen in regards to each biological endpoint. At 0.1 μM analog (2), cell viability was not different from the untreated control (FIG. 9B), as compared to the 60% cell death observed at the equivalent concentration of triptolide.

However, if cells were allowed to continually grow at 0.1 μM analog (2) out to 72 hours, it became evident that there was an overall growth suppressive effect. Interestingly, 1 μM of analog (2), a concentration that efficiently competes with triptolide for binding (FIG. 3B) induced greater than 50% cell death while having no effect on NFκB transcriptional activity (FIG. 9B). It therefore appears that the mechanism of NFκB inhibition is more sensitive to the structural integrity of the 12,13 epoxide than is the cell growth/death regulatory pathway.

The most potent and biologically similar to triptolide was analog (3). Displacement of [³H]-triptolide binding was near complete at both the 1 and 10 μM concentration (FIG. 3B). In fact, 1 μM analog (3) actually elicited a higher competitive ability than triptolide itself at the same concentration (FIG. 3B). Both profiles of cell death and transcriptional repression mimicked triptolide with no significant difference from 0.1-10 μM (FIG. 9C). It is of note however that at 25 nM, a concentration shown to induce cell death by triptolide (FIG. 8A), analog (3) had only a growth suppressive effect. Since competition for binding by analog (3) was so strong, this data would support the idea that triptolide induced apoptosis at its lowest (25 nM) concentration is partially due to the functionality of the C-14 hydroxyl.

In sum, triptolide has a broad range of therapeutic potentials ranging from attenuation of inflammation, suppression of auto-immunity, and the elimination or regression of certain tumors. Basic studies of triptolide's mechanisms of action are incomplete with little understanding of how this small molecule can elicit such a broad range of effects. Utilizing [³H]-triptolide as a probe, Applicant examined the properties of triptolide binding in the cell as well as addressed questions pertaining to two well described biological endpoints of triptolide function: cell death and transcriptional repression of NFκB. A specific triptolide binding activity is present within intact cells, is reversible, associates predominantly with cellular membranes, and is sensitive to calcium levels. While triptolide binding increases upon extracellular calcium depletion, it is severely impaired in its ability to induce cell death. This observed calcium dependence is specific to the regulation of apoptosis as triptolide's effect on NFκB transactivation is unaltered in the presence or absence of calcium. An overall separation of biological effects can be further discerned when triptolide is present at low nanomolar concentrations. While 10 nM is growth inhibitory and 25 nM induces cell death, neither of these concentrations can elicit transcriptional repression. Limited structure-function analysis utilizing triptolide analogs has demonstrated that while competitive binding for triptolide interaction sites is intact, biological effects are highly dependent upon structural moieties. These findings implicate triptolide as functioning through at least two separable pathways distinguishable by calcium requirements, sensitivity to drug concentration and preference towards structural entities.

7. Experimental Procedures.

A) Reagents

Triptolide was obtained from Sinobest Inc. (China) and purity was 99% as determined by HPLC. DMSO was used to dissolve triptolide and was then directly added into culture media for all experiments. Triptolide was tritiated by Sib Tech, Inc. (Newington, Conn.) and resuspended in ethanol to a specific activity of 4-6 Ci/mmol. Purity was >95% as confirmed by RP-HPLC on a Hypersil C18 column and by TLC on both C18 and silica gel. Epi-Triptolide/Triol-Triptolide (C.A.S, No 147852-78-6), and Triptonide (C.A.S, No 38647-11-9) were purchased from Sequoia Research Products (United Kingdom).

B) Cell Culture and Viability Studies

HeLa cells were incubated in DMEM or SMEM (Gibco) media+10% FBS and maintained at 37° C. in 5% CO₂ for all experiments. HeLa cell viability was assessed by trypan blue dye exclusion, as well as by morphological examination (non-viable cells were rounded and detached from culture plate).

C) [³H]-Triptolide Labeling of HeLa Cells

Labeling studies were performed by the addition of approximately 30 nM [³H]-triptolide directly into the culture media for one hour at 37° C. For cold competition studies, 1 μM triptolide was incubated with the cells for one hour before or after the addition of [³H]-triptolide. Triptolide analog studies followed a similar protocol where concentrations used for competition were either 1 or 10 μM added before [³H]-triptolide. Medium was removed and cells were washed 3× in cold PBS. Total cell lysates were prepared (150 mM NaCl, 50 mM Tris-HCl pH 7.4, 1 mM EDTA, 1% Triton X-100, and Complete™ protease inhibitors (Roche)) and protein was quantitated before measuring the [³H]-triptolide binding activity via liquid scintillation.

DE-52 anion exchange resin (Whatman, Inc.), a diethylaminoethyl (DEAE)-cellulose, was prepared for binding by a 1 M sodium chloride (NaCl) wash followed by multiple washes with 0 M salt buffer (10 mM HEPES pH7.4, 0.1 mM EDTA, 1 mM DTT, 0.1% Triton X-100). HeLa cell lysates labeled with [³H]-triptolide were passed over the resin and allowed to bind at 4° C. for 30 minutes before collecting the flowthrough and subsequent washes. A step gradient from 0.0 to 1.0 M NaCl was used for protein elution. All fractions were subsequently counted by liquid scintillation.

For cellular fractionation studies, cells were allowed to swell on ice and lysed by passage through a syringe in a hypotonic lysis buffer (10 mM Tris-HCl+complete protease inhibitors). The lysate was centrifuged at 100,000×g and the supernatant was saved as the S-100 cytosolic fraction. The pellet was washed and resolubilized in 1% Triton X-100 containing lysis buffer. Following centrifugation, the supernatant was saved as the P-100 membrane fraction. Additionally, P-100 lysates were run out under native or reducing gel conditions without boiling. Gel slices were measured out in equal increments and the molecular weight range of each was calculated. Each gel piece was crushed in ddH₂O followed by scintillation counting of the water extract.

D) [³H]-Triptolide Specific Binding

Saturation binding assays were accomplished in HeLa cells adhered on 6-well plates in DMEM+10% FBS. All samples were at least 90% confluent at time of addition of triptolide. Non-specific binding of [³H]-triptolide was assessed by the pre-incubation of 2 μM (non-labeled) triptolide for one hour. Following cold competition (or DMSO) 5, 10, 20, 50, or 100 nM of [³H]-triptolide was added into the cultures for an additional one hour, and then cells were lysed and counted for binding activity.

E) Transfection of Parvalbumin Constructs

All parvalbumin-GFP constructs and control vectors (Pusl, et al., 2002, J Biol Chem 277, 27517-27527) were a gift of Anton Bennett (Yale University). Hela cells were plated out on 6- or 12-well plates at a density of 5×10⁵ or 1×10⁵, respectively. Cells were transiently transfected with 0.5-1 μg of one of the following pcDNA3 derived plasmids for 24 hours in DMEM/10% FBS+Lipofectamine 2000 (Invitrogen): CMV-parvalbumin-GFP, CMV-NES-parvalbumin-GFP, or CMV-NLS-parvalbumin-GFP. Following confirmation of GFP expression and localization by microscopy, 100 nM triptolide was added into each transfected cell population (>90% transfection efficiency). Cell viability was assessed at 24 and 48 hours post triptolide addition by morphology and trypan blue dye exclusion.

F) NF-kappa B luciferase assay

A triple κB promoter-Luciferase reporter construct was a gift of Sankar Ghosh (Yale University). HeLa cells were plated at a density of 2×10⁵ in 12 well plates and transfected with 100 ng of the KB-Luciferase plasmid plus Lipofectamine 2000 (Invitrogen) for 24 hours before the addition of 100 nM triptolide for one hour and 15 ng/ml of recombinant (human) TNF-α (Roche) for an additional five hours. The transfection efficiency of HeLa was determined to be 80-90%, and all samples were normalized to protein concentration. Luciferase assays were performed using the Firefly luciferase kit as per manufacturer's protocol (Promega) and results obtained on the Wallac Victor2 1420 Multilabel Counter (Perkin Elmer).

### Example 8

### Triptolide Related Compounds Attenuate Polycystic Disease Progression Mediated by Polycystin-2

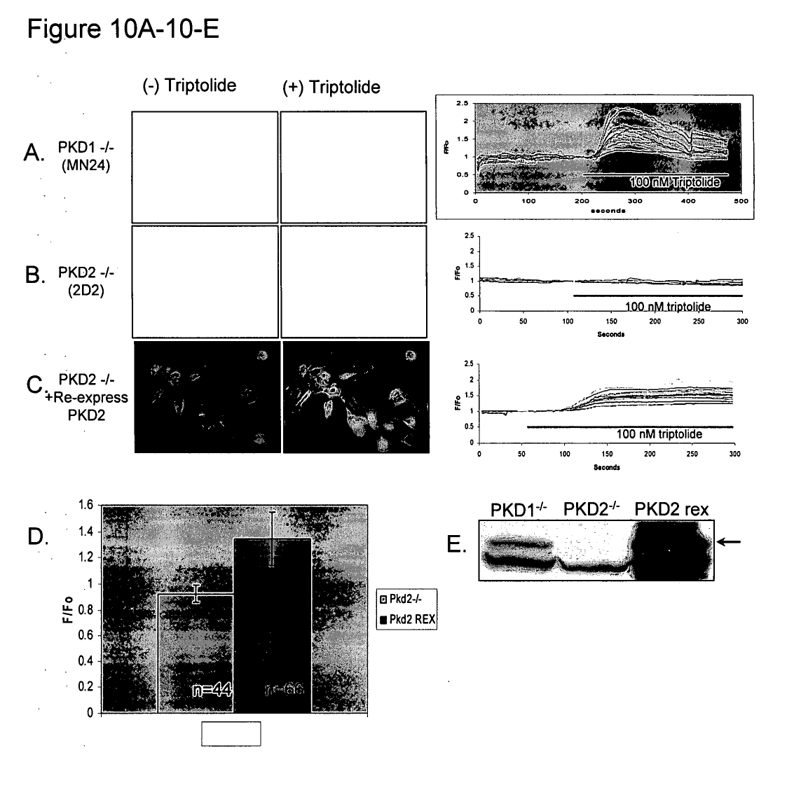

Murine kidney epithelial cell lines with differing polycystin-1 or polycystin-2 expression were used to establish a cellular based mechanism for polycystin-2 mediated calcium release in response to triptolide. Because the biochemical purification analysis identified polycystin-2 as a putative triptolide binding protein, Applicant assessed whether the calcium release was dependent upon expression of polycystin-1. Epithelial cells derived from the proximal nephric tubules of Pkd1^{-/-} mice were first examined to determine if calcium release was observed when 100 nM of triptolide was perfused through the imaging chamber. There was a clear rise in intracellular calcium levels upon triptolide addition, demonstrating that triptolide was capable of eliciting calcium release in cells (FIG. 10A); and furthermore, that this biological activity was not dependent upon polycystin-1 expression. When the identical system was used to perfuse 100 nM triptolide over Pkd2^{-/-} murine kidney epithelial cells, no calcium release was detected (FIG. 10B). To strengthen the evidence that polycystin-2 was necessary for calcium release elicited by triptolide addition, it was reconstituted by stable expression of Pkd2 into the background of Pkd2^{-/-} cells, which were assessed for sensitivity to triptolide. Re-expression of polycystin-2 restored calcium release in this cell line, providing mechanistic evidence for triptolide-mediated calcium regulation (FIG. 10C).

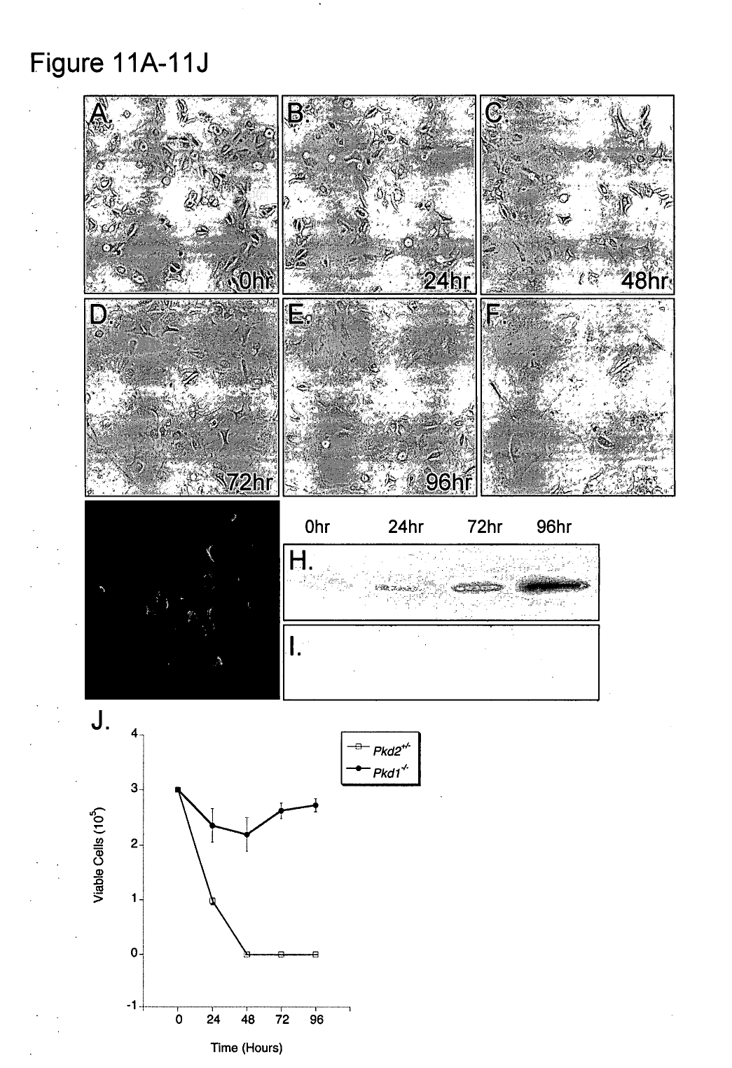

Calcium response to triptolide was, thus, shown to be dependent on polycystin-2. The biological response to calcium flux was next assessed in the murine Pkd1^{-/-} cell line, by adding 100 nM triptolide to the cultured cells and observing cell growth over time. Within the first 24 hours of culture, a minimal number of detached cells was observed. Over 96 hours, the remaining cells were growth arrested, as evidenced by their flattened morphology and the fact that the overall cell number did not increase (FIG. 11F). In the absence of triptolide, this cell line underwent a population doubling every 48 hours. In contrast, murine cell lines expressing at least one copy each of Pkd1 and Pkd2 (i.e., Pkd2+/−) underwent rapid cell death within 24 hours, suggesting a more potent role for triptolide when both proteins are expressed and can associate (FIG. 9J). It is possible that additional signaling pathways are activated by triptolide when Pkd1 is expressed. PKD1^{-/-} cells have previously been shown to down-regulate p21 expression as they proliferate (Bhunia, et al., 2002, Cell, 109:157-168). Therefore, Applicant assessed whether the inhibition of proliferation observed was due to p21 re-expression upon triptolide treatment. Over a 96 hour time course it became apparent that p21 was upregulated in the triptolide treated population, thereby re-establishing the normal state of growth arrest in these kidney epithelial cells (FIG. 11H). The presence of active caspase-3 was assessed by western blot analysis and again the results failed to implicate triptolide induced apoptosis in the Pkd1−/− cell line (FIG. 11H). Thus, these in vitro data indicate that triptolide is capable of eliciting a polycystin-2 mediated calcium release, which results in p21 up-regulation and inhibition of Pkd1^{-/-} cell proliferation.

ADPKD is thought to result from a defect of calcium signaling due to the loss of the mechanosensory function of the primary cilia (Nauli, et al., 2003, Nat Genet 33, 129-137). Therefore, Applicant sought to determine whether triptolide could artificially restore calcium flux in the Pkd1^{-/-} mouse model and arrest or delay the proliferative cystic state. Pkd1^{-/-} animals are not viable, although pups may develop to a late gestational state (E18.5-19.5). Such animals exhibit severe developmental abnormalities, such as cardiovascular (Boulter, et al., 2001, Proc Natl Acad Sci USA 98, 12174-12179; Kim, et al., 2000, Proc Natl Acad Sci USA 97, 1731-1736) and skeletal defects (Boulter, et al., 2001, Proc Natl Acad Sci USA 98, 12174-12179; Lu, et al., 2001, Hum Mol Genet 10, 2385-2396), in addition to kidney and pancreatic cyst formation (Wu, et al., 2002, Hum Mol Genet 11, 1845-1854; Lu, et al., 1997, Nat Genet 17, 179-181). Therefore, rescue from lethality seemed unlikely. Kidney cysts begin to form on E15.5 in the proximal tubules and rapidly progress into the cortex (Lu, et al., 1997, Nat Genet 17, 179-181). In Pkd1^{-/-} E18.5-19.5 pups, large kidney cysts are readily apparent upon gross morphological examination, as well as by histological staining.

Triptolide has been previously studied in rodent models of tumor regression (Tengchaisri, et al., 1998, Cancer Lett 133, 169-175; Yang, et al., 2003, Mol Cancer Ther 2, 65-72), but it had not yet been tested in a system utilizing pregnant females. To first establish a potential therapeutic versus lethal concentration of drug delivery, pregnant C57B1/6 mice were treated with incremental concentrations of triptolide between 0.01-0.15 mg/kg/day i.p. injections. Toxicity was assessed as determined by resorption of all embryos or the preponderance of a large percentage of stillborns. With reference to these criteria, triptolide toxicity was determined to be most prominent at concentrations of 0.1 mg/kg/day or greater. However, no discernable adverse effects were observed at a dosage of 0.07 mg/kg/day, which was used as the maximum tolerated dose. Another experimental parameter involved the timing of the start of triptolide injections, since polycystin-2 has been implicated in left-right axis formation in the developing embryo at approximately E7.75 (McGrath, et al., 2003, Cell 114, 61-73; Pennekamp, et al., 2002, Curr Biol 12, 938-943). Applicants therefore chose E10.5 as the start of triptolide injections, in order to allow for a normal polycystin-2 mediated patterning event and still leave sufficient time to act on cyst formation during kidney organogenesis.

Following successful Pkd1^{+/-} Pkd1^{+/-} matings, 0.07 mg/kg/day of triptolide or DMSO control was injected i.p. into pregnant mice until they gave birth. All pups were assessed for viability, length, developmental staging, and wet kidney weight. A total of 59 pups from DMSO treated females and 100 pups from triptolide treated females were examined for multiple parameters, such as genotypic distribution, developmental stage at time of birth, and average kidney weights (Table 1). It has been previously demonstrated that Pkd1^{-/-} mice can be reabsorbed beginning at E12.5, due to the severe edema, vasculature defects and abnormal skeletogenesis, thereby resulting in an atypical Mendelian distribution of Pkd1^{-/-} progeny (Wu, et al., 2002, Hum Mol Genet. 11, 1845-1854; Lu, et al., 2001, Hum Mol Genet 10, 2385-2396). The same reported deviation was observed in expected Pkd1^{-/-} numbers, with 20% and 18% for DMSO or triptolide treatment, respectively (Table 1). Approximately 20% of all Pkd1^{-/-} mice were born alive from each treatment group. However, severe edematous abnormalities were obvious upon necropsy. Independent of genotype, triptolide did not have any apparent overall deleterious effect on murine development or length of pregnancy.

TABLE 1
Descriptive Summaries for DMSO or Triptolide Treated Mice.

| | DMSO | 0.07 mg/kg/day Triptolide |
|---|---|---|
| Litters | 7 | 15 |
| Total Pups | 59 | 100* |
| Total Pkd1^{+/+} | 20 (34%) | 33 (33%) |
| Total Pkd1^{+/-} | 27 (46%) | 46 (46%) |
| Total Pkd1^{-/-} | 12 (20%) | 18 (18%) |
| Alive Pkd1^{-/-} (birth) | 2 (17%) | 4 (22%) |
| Ave. length Pkd1^{-/-} (mm) | 24.8 ± 0.4 | 24.6 ± 0.8 |
| Ave. kidney wet | 8.0 ± 0.4 | 7.2 ± 0.2 |
| weight Pkd1^{+/+} (mg) | | |
| Ave. kidney wet | 7.5 ± 0.2 | 8.0 ± 0.3 |
| weight Pkd1^{+/-} (mg) | | |
| Ave. kidney wet | 17.5 ± 2.0 | 21.8 ± 2.8 |
| weight Pkd1^{-/-} (mg) | | |
| Ave. Delivery date | 19.4 ± 0.3 | 19.5 ± 0.3 |
| (Embryonic day) | | |

*3 are of unknown genotype, averages are presented as mean ± SE

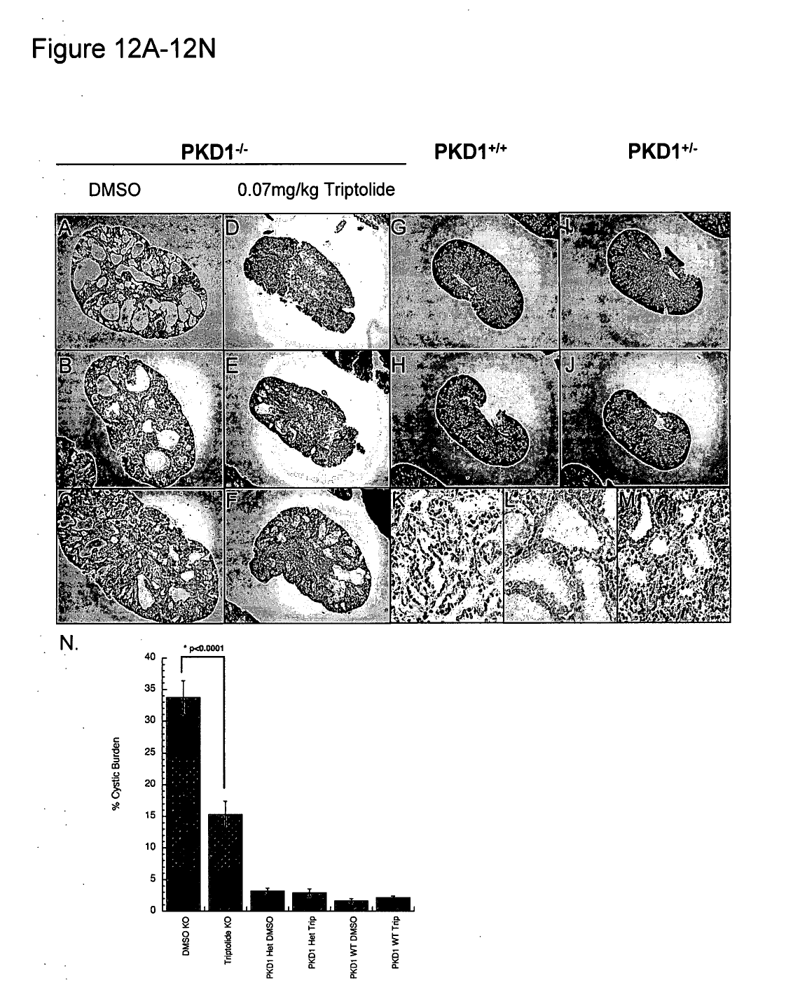

Initial examination of kidney pathology was by gross morphology. Pkd1^{-/-} kidneys, on average, were larger and in some cases cyst formation could be readily visualized. Wet weight kidney analysis from Pkd1^{+/+} or Pkd1^{+/-} mice demonstrated no significant difference in weight or overall size for DMSO or triptolide treated, respectively (Table 1). Pkd1^{-/-} kidneys were larger by weight, although there was no difference between DMSO or triptolide treatment (24.8±0.4 vs. 24.6±0.8 mg), indicating fluid secretion was not affected. Sagittal cross-sectioning of kidneys, H&E staining and the calculation of the area of cyst formation as a percentage of total kidney area was completed for each sample. Since the in vitro data have shown that the expression of both polycystin-1 and -2 results in cell death from triptolide treatment, it was possible that normal kidney development may have been adversely affected. This was not the case: Pkd1^{+/+} and Pkd1^{+/-} kidneys in both treatment groups showed normal morphology where background “cyst values” were calculated to account for random physiological abnormalities or artifacts of tissue handling and preparation. Pkd1^{-/-} kidneys from animals injected with DMSO had a mean cystic burden of 34±2.7%; several had cystic masses between of 55-65% of the whole kidney (FIG. 12A-C).

Triptolide treatment during the gestation of Pkd1^{-/-} pups resulted in a statistically significant decrease in the cystic burden to an average of 15±2.1% (FIG. 12D-F). There was some litter variability where there was a range from small kidneys with almost no evidence of any cyst formation, to a maximum cyst burden of 25%. This variability may be due to factors such as the proximity of triptolide delivery to the developing fetus during injections and difficulty in providing an effective therapeutic dose of triptolide while avoiding toxicity. The epithelial cells lining the cysts looked normal by microscopy and the diameter of cyst lumens on average was smaller. However, Applicant wanted to determine if the lack of cyst growth due to triptolide treatment was due to the induction of apoptosis or a delay in cell growth. To complement the in vitro data, tissue sections were stained for immunoreactivity towards active caspase-3, a marker of cellular commitment to apoptosis. Both DMSO (FIG. 12L) and triptolide (FIG. 12M) treated samples did not show any significant activation of the caspase pathway, as determined by comparison to secondary antibody staining alone (FIG. 12K). This is an indication the apoptotic pathway was not activated.

ADPKD cyst formation may be likened to benign epithelial neoplasia, in that both are characterized by uncontrolled cellular proliferation, independent of extracellular cues. Triptolide has been investigated for many of its potential therapeutic uses, including reduction of solid tumor masses, and is currently in clinical trials for its potent effect in a prostate cancer model (Kiviharju, et al., 2002, Clin Cancer Res 8, 2666-2674). In this respect, triptolide has been shown repeatedly to induce efficient apoptosis or cell growth arrest; the effect that results is dependent upon the effective concentration of the drug. Until now, upstream targets of triptolide efficacy have not been elucidated that explain its broad and potent biological effects. Furthermore, the discovery by our laboratory that polycystin-2 is required for triptolide mediated calcium release correlates with previous findings that triptolide binding and cell death or growth arrest can be modulated by calcium concentration (see, e.g., Example 7).

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or aiding in the treatment of polycystic kidney disease (PKD) in an individual in need thereof, comprising administering to the individual a therapeutically effective amount of a polycystin-2 (PC-2) agonist.

2. The method of claim 1, wherein the PC-2 agonist regulates PC-2-mediated calcium signaling in kidney cyst tissues.

3. The method of claim 1, wherein the PC-2 agonist is a small molecule.

4. The method of claim 1, wherein the PC-2 agonist is a triptolide-related compound.

5. The method of claim 4, wherein the triptolide-related compound is triptolide or a triptolide prodrug or a triptolide derivative selected from triol-triptolide triptonide 14-methyl-triptolide, 14-deoxy-14α-fluoro-triptolide, 5α-hydroxy triptolide, 19-methyl triptolide, and 18-deoxo-19-dehydro-18-benzoyloxy-19-benzoyl triptolide, and 14-acetyl-5,6-didehydro triptolide.

6. (canceled)

7. (canceled)

8. The method of claim 1, further comprising administering to the individual a second therapeutic agent for treating PKD.

9. The method of claim 8, wherein the second therapeutic agent is selected from an EGF receptor kinase inhibitor, a cyclooxygenase 2 (COX2) inhibitor, a vasopressin V₂ receptor inhibitor, a ligand of a peripheral-type benzodiazepine receptor (PTBR), a somatostatin analogue (e.g., octreotide), rapamycin and pioglitazone.

10. (canceled)

11. (canceled)

12. (canceled)

13. (canceled)

14. (canceled)

15. (canceled)

16. (canceled)

17. (canceled)

18. A method of treating or aiding in the treatment of a condition caused by abnormal calcium signaling, comprising administering to an individual in need thereof a therapeutically effective amount of a PC-2 agonist.

19. (canceled)

20. (canceled)

21. (canceled)

22. A method of treating a cystic disease in an individual in need thereof, comprising administering to the individual a therapeutically effective amount of a PC-2 agonist in an amount sufficient to slow or inhibit growth of cyst cells.

23. (canceled)

24. The method of claim 22, wherein the individual has or is at risk of developing PKD.

25. A method of slowing or inhibiting cyst formation, comprising contacting cyst cells with a PC-2 agonist in an amount sufficient to slow or inhibit growth of cyst cells.

26. The method of claim 25, wherein the cyst cells are in an individual having or at risk of developing a cystic disease.

27. A method of regulating calcium influx in a cell expressing polycystin-2, comprising contacting the cell with an effective amount of a PC-2 agonist.

28. The method of claim 27, wherein the cell is a kidney cell or a bile duct cell or a pancreatic duct cell.

29. (canceled)

30. A method of identifying a PKD2 agonist, comprising:
(a) contacting a test agent with a cell expressing PC-2;
(b) measuring PC-2-mediated calcium release in the cell; and
(c) comparing the level of PC-2-mediated calcium release obtained in (b) with the level obtained under the same conditions but in the absence of the test agent,
wherein a greater level of PC-2-mediated calcium release in the presence of the test agent than in the absence of the test agent indicates that the test agent is a PC-2 agonist.

31. (canceled)

32. A method of identifying a therapeutic agent for slowing or inhibiting cyst formation, wherein cyst formation is associated with inadequate PC-1 function/activity in an individual, comprising:
(a) contacting a test agent with a cell expressing PC-2, but not PC-1;
(b) measuring PC-2-mediated calcium release in the cell; and
(c) comparing the level of PC-2-mediated calcium release obtained in (b) with the level obtained under the same conditions but in the absence of the test agent, wherein a greater level of PC-2-mediated calcium release in the presence of the test agent than in the absence of the test agent indicates that the test agent is a therapeutic agent for slowing or inhibiting cyst formation.

33. (canceled)

34. (canceled)

35. (canceled)

36. A method of identifying a PC-2 agonist, comprising
(a) contacting cells that express PC-2 and lack adequate levels of functional PC-1 with a candidate PC-2 agonist, under conditions appropriate for PC-2 mediated increase in calcium inside cells to occur;
(b) assessing calcium levels inside the cells;
(c) comparing calcium levels in the cells assessed in (b) with calcium in corresponding cells, under the same conditions as in (a) but in the absence of the candidate PC-2 agonist;
(d) comparing the calcium levels in cells assessed in (b) with the calcium levels in cells under the conditions as in (a) that lack adequate levels of functional PC-2 and PC-1; and
(e) comparing the calcium levels in cells assessed in (b) with the calcium levels in cells, maintained under the same conditions as in (a), that lack adequate levels of functional PC-2 but express adequate functional PC-1, wherein if there is a greater increase in calcium inside the cells in (a) than in the corresponding cells in the absence of the candidate PC-2 agonist and in the cells in (d) and/or (e), the candidate PC-2 agonist is a PC-2 agonist.

37. The method of claim 36, wherein the cells are epithelial cells, kidney epithelial cells or a kidney epithelial cell line.

38. The method of claim 37, wherein the cells have formed cilia.

39. (canceled)

40. (canceled)

41. (canceled)

42. (canceled)

43. (canceled)

44. The method of claim 36, wherein cells contacted with the candidate PC-2 agonist have incorporated therein a calcium sensitive fluorescent dye and calcium increase in cells is assessed by measuring fluorescence of cells prior to being contacted with the candidate PC-2 agonist and after being contacted with the candidate PC-2 agonist, wherein if fluorescence of cells is greater after being contacted with the candidate PC-2 agonist than before being contacted with the candidate PC-2 agonist, the candidate PC-2 agonist is a PC-2 agonist.

45. The method of claim 36, further comprising administering a PC-2 agonist identified by the method to an appropriate animal model of ADPKD and assessing effects of the PC-2 agonist on cyst formation and/or reversal in the animal model.

46. A method of identifying a therapeutic agent which is a PC-2 agonist for administration to an individual who has or is at risk of having ADKPD, comprising:
(a) contacting cells that express PC-2 and lack adequate levels of functional PC-1 expression with a candidate PC-2 agonist, under conditions appropriate for PC-2 mediated increase in calcium in the cells to occur;
(b) assessing calcium increase in the cells;
(c) comparing calcium increase in the cells assessed in (b) with calcium increase in corresponding cells, under the same conditions as in (a) but in the absence of the candidate PC-2 agonist;
(d) comparing the calcium levels in cells assessed in (b) with the calcium levels in cells under the conditions as in (a) that lack adequate levels of functional PC-2 and PC-1; and
(e) comparing the calcium levels in cells assessed in (b) with the calcium levels in cells, maintained under the same conditions as in (a), that lack adequate levels of functional PC-2 but express adequate functional PC-1,
wherein if there is a greater increase in calcium inside the cells in (a) than in the corresponding cells in the absence of the candidate PC-2 agonist, and in the cells in (d) and/or (e) the candidate PC-2 agonist is a PC-2 agonist.

47. The method of claim 46, wherein the cells are epithelial cells, kidney epithelial cells or a kidney epithelial cell line.

48. The method of claim 47, wherein the cells have formed cilia.

49. (canceled)

50. (canceled)

51. (canceled)

52. (canceled)

53. (canceled)

54. The method of claim 46, wherein cells contacted with the candidate PC-2 agonist have incorporated therein a calcium sensitive fluorescent dye and calcium uptake or release from intracelluar stores into cells is assessed by measuring fluorescence of cells prior to being contacted with the candidate PC-2 agonist and after being contacted with the candidate PC-2 agonist, wherein if fluorescence of cells is greater after being contacted with the candidate PC-2 agonist than before being contacted with the candidate PC-2 agonist, the candidate PC-2 agonist is a PC-2 agonist.

55. The method of claim 46, further comprising administering a PC-2 agonist identified by the method to an appropriate animal model of ADPKD and assessing effects of the PC-2 agonist on cyst formation and/or reversal in the animal model.

56. A therapeutic method for treating or preventing ADKPD in an individual who has or is at risk of having ADKPD, comprising administering to the individual a therapeutic amount of a PC-2 agonist identified by the method of claim 46.

57. (canceled)

58. The method of claim 56, further comprising administering a second therapeutic agent for treatment of ADPKD, which is not a PC-2 agonist, to the individual.

59. (canceled)

60. (canceled)

61. An isolated peptide that localizes to cilia of kidney tubule epithelial cells, comprising the first 15 amino acid residues of polycystin-2 or an equivalent of the isolated peptide.

62. The isolated peptide of claim 61, wherein the polycystin-2 is H. sapien PC-2; M. musculus PC-2; R. norvegicus PC-2; D. rerio PC-2; or S. purpuratus PC-2.

63. The isolated peptide of claim 61, comprising the motif R₆VXP.

64. A peptide that localizes to cilia of kidney tubule epithelial cells, referred to as a trafficking peptide, linked to a heterologous protein that, in the absence of the trafficking peptide, does not localize to cilia of kidney tubule epithelial cells.

65. The peptide of claim 64, wherein the peptide that localizes to cilia comprises the first 15 amino acid residues of polycystin-2 or an equivalent thereof.

66. The peptide of claim 64, wherein the 15 amino acid residues are those present in human PC-2, wherein the 15 amino acid residues are MVNSSRVQPQQPGDA (SEQ ID 1).

67. The peptide of claim 64, wherein the peptide comprises the amino acid motif represented by R₆VXP.

68. (canceled)

69. (canceled)

70. (canceled)

71. (canceled)

72. The pharmaceutical composition of claim 71, additionally comprising a second therapeutic agent that is not a PC-2 agonist.

73. A method of identifying a selective PC-2 agonist, comprising:
(a) contacting cells that express PC-2 and lack adequate levels of functional PC-1 with a candidate PC-2 agonist, under conditions appropriate for PC-2 mediated increase in calcium inside the cells to occur;
(b) assessing calcium levels or concentrations inside cells contacted in (a);
(c) contacting cells that do not express PC-2 and lack adequate levels of functional PC-1 with a candidate PC-2 agonist, under conditions appropriate for PC-2 mediated increase in calcium inside the cells to occur;
(d) assessing calcium levels or concentrations inside cells contacted in (c); and
(e) comparing calcium levels or concentrations in cells assessed in (b) with calcium levels or concentrations in cells assessed in (d),
wherein if there is a greater increase in calcium levels or concentrations inside cells contacted in (a) than in cells contacted in (c) (e.g., if the level or concentration of calcium in cells is greater in cells that express PC-2 than in cells that do not express PC-2), the candidate PC-2 agonist is a selective PC-2 agonist.
